(19) United States
(12) Patent Application Publication
(10) Pub. No.: US 2003/0072743 A1
(43) Pub. Date: Apr. 17, 2003

(54) Genetic manipulation of phagocytes for modulation of antigen processing and the immune response therefrom

(76) Inventors: Matthew Albert, New York, NY (US); Raymond Birge, New York, NY (US)

Correspondence Address: KLAUBER & JACKSON, 411 HACKENSACK AVENUE, HACKENSACK, NJ, 07601

(21) Appl. No.: 10/238,213

(22) Filed: Sep. 10, 2002

Related U.S. Application Data
(63) Continuation of application No. 09/565,958, filed on May 5, 2000, now abandoned.

(51) Int. Cl. A61K 48/00; C12N 5/08; C07K 14/74

(52) U.S. Cl. 424/93.21; 435/372; 530/350

(57) ABSTRACT

The present invention is directed to methods for enhancing the ability of the immune system to either increase or decrease a cellular immune response to an antigen, for the purpose of either enhancing effectiveness of, for example, anti-viral and anti-tumor responses or decreasing immunological reactions in, for example, autoimmune disease or organ rejection, respectively; or clearing certain antigens responsible for disease in order to prevent an immune response.

### GOVERNMENTAL SUPPORT

The research leading to the present invention was supported, at least in part, by a grant from the U.S. Public Health Service, National Institutes of Health, Grants No. GM-07793 and GM-55760. Accordingly, the Government may have certain rights in the invention.

### CROSS-REFERENCE TO RELATED APPLICATION

This application claims priority to U.S. application Ser. No. 09/251,896, filed Feb. 19, 1999, and is incorporated herein by reference in its entirety.

### FIELD OF THE INVENTION

The present invention is directed to methods for enhancing the ability of the immune system to either increase or decrease a cellular immune response to an antigen, for the purpose of either enhancing effectiveness of, for example, anti-viral and anti-tumor responses or decreasing immunological reactions in, for example, autoimmune disease or organ rejection, respectively; or clearing certain antigens responsible for disease in order to prevent an immune response.

### BACKGROUND OF THE INVENTION

The initiation of CD8+ T cell (cytotoxic T cell, or CTL) immunity requires the presentation of processed antigens by antigen presenting cells (APCs) (1). T cells recognize fragments of antigens bound to class I major histocompatibility complex (MHC) molecules on the surface of an APC. Classically, peptides that bind class I MHC molecules are derived from proteins that are synthesized endogenously within the cell (e.g. self and viral proteins) (2). This presumes that direct infection of the APC is a pre-requisite for developing immunity to viruses; and excludes the possibility of generating immunity to tumor-restricted antigens. There is a growing body of evidence, however, which suggests that exogenous antigens, which should not gain access to the cytoplasm, can be channeled into the endogenous pathway of an APC for MHC I presentation.

The most compelling data comes from in-vivo experiments in murine models demonstrating that viral, tumor and minor histocompatibility antigens can be transferred from donor cells to host bone marrow derived APCs to elicit antigen-specific CTL responses (3-6). This phenomenon has been referred to as “cross-priming,” and the processing of antigen for T cell presentation as “cross-presentation” (3). These observations indicate that the immune system has a natural mechanism by which exogenous antigens may access the MHC I antigen presentation pathway of an APC. However, there were two undefined features: the mechanism by which the antigens are acquired (whole cells vs. free protein or peptide) and the identity of the APC.

As described, for example, in co-pending International Application PCT/US99/03763 (WO 99/42564), dendritic cells (DCs) have been found to be the APCs responsible for mediating cross-presentation; and as shown in PCT/US99/03763, apoptotic cells serve to deliver the exogenous antigen in a manner which permits class I antigen presentation (7, 8, 9).

Apoptosis is now widely recognized as the primary mechanism whereby physiologic cell death occurs. In vivo, the typical fate for such apoptotic cells is rapid engulfment and degradation by phagocytes (10-12). In various in-vitro systems, it has been shown that the phagocyte engages and internalizes the dying cells via various surface receptors (13, 14). In this way, dying cells, which contain potentially inflammatory factors, are rapidly cleared by neighboring cells, scayenger cells, or macrophages, without inducing an inflammatory response. Additionally, it has been suggested that immature DCs phagocytose apoptotic cells via the β₅ integrin receptor, a receptor restricted to DCs as compared to macrophages, the latter being an APC capable of capturing apoptotic cells, but unable to cross-present antigen (15).

Recently, however, it has been demonstrated that this process is not as immunologically quiescent as once believed. Following the phagocytosis of apoptotic cells, macrophages release significantly less of the pro-inflammatory cytokines IL-1, TNF-α and IL-12, now modulating their response toward immunosuppressive factors such as IL-10 and TGF-β (16, 17). Dendritic cells (DCs), in contrast, can generate peptide epitopes derived from antigen within the engulfed apoptotic cells, and stimulate antigen-specific class I-restricted CD8+ T cells (7). This latter finding led to the discovery of a novel pathway for the generation of MHC I/peptide complexes and has helped define a mechanism by which the in-vivo phenomenon of “cross-priming” and “cross-tolerance” might occur (reviewed in 18).

Dendritic cells (DCs) handle apoptotic material in a unique matter. DCs in the periphery exist as immature cells, where they serve as “sentinels” (19), responsible for capturing antigen (reviewed in 20), including apoptotic cells (e.g. the phagocytosis of tumor cells undergoing apoptosis [21]). Upon activation/maturation, DCs migrate to the draining lymph organs, where they are may initiate an immune responses (22, 23). This ability to traffic out of peripheral tissue with captured antigen, and enter the afferent lymph is unique to the DCs, making them the appropriate carrier of tissue-restricted antigen to lymph organs for the initiation of viral- and tumor-immunity.

Exploiting the recently-described phenomenon of apoptotic-cell delivery of antigen may provide an opportunity to stimulate the various functions of the immune system to achieve more rapid and/or robust therapeutic goals, whether enhancement or suppression of the immune response, or degradation of an antigen. It is towards the enhancement of the modulation of the immune response by apoptotic cell-delivered antigens that the present invention is directed.

### SUMMARY OF THE INVENTION

In its broadest aspect, the present invention is directed to a method for enhancing the ability of a phagocyte to capture at least one apoptotic-cell-delivered antigen or altering the trafficking of the internalized apoptotic material by genetically modifying the phagocyte to express or increase its expression of a receptor which facilitates the capture of apoptotic cells. This may be achieved, for example, by genetically modifying phagocytes to express at least one apoptotic-cell receptor, by genetically modifying phagocytes to increase the expression of at least one endogenous apoptotic-cell receptor, or by genetically modifying phagocytes to express a modified apoptotic cell receptor with enhanced affinity for apoptotic cells. Apoptotic-cell receptors useful for these purposes include but are not limited to a member of the Fc receptor family, a member of the scavenger receptor family, CD14, a member of the ABC-1 family of transporters of transporters, a member of the C-type lectin family, an integrin receptor β subunit other than β₁, an integrin receptor heterodimer other than that comprising β₁, an integrin receptor heterodimer comprising a chimeric β subunit other than β1, or an integrin receptor heterodimer comprising a mutant β subunit with signaling properties similar to β₅. For example, the integrin receptor β subunit may be β₅ or the integrin receptor heterodimer may be αᵥβ₅. The integrin receptor heterodimer that comprises a chimeric β subunit may be a wild-type α subunit and a chimeric β subunit, wherein the chimeric β subunit may be an extracellular integrin receptor β domain fused with a signaling domain derived from a molecule including but not limited to an integrin receptor β subunit other than β₁, a member of the Fc receptor family, a member of the scavenger receptor family, or a member of the C-type lectin family. By way of example, the signaling domain derived from a member of the Fc receptor family may be the FcγRI, FcγRIIA, FcγRIIB or FcγRIII α-chain or the signaling sequence of the Fc γ-chain; the signaling domain derived from an integrin receptor β subunit other than β₁ may be that of β₂, β₃ or β₅.

The phagocyte may be, by way of non-limiting example, a professional phagocyte or a nonprofessional phagocyte. Examples of professional phagocytes include but are not limited to antigen presenting cells, macrophages, B cells, and neutrophils. The antigen presenting cells may be, for example, a dendritic cell, such as a myeloid or a lymphoid dendritic cell. Nonlimiting examples of nonprofessional phagocytes include keratinocytes, epithelial cells, fibroblasts, and endothelial cells. The phagocyte may be a human phagocyte or a non-human phagocyte.

Genetically modifying the phagocyte to express or increase expression of at least one apoptotic cell receptor may be carried out by any number of means for introducing genetic material into a cell that is subsequently expressed. By way of non-limiting examples, such methods generally and specifically include infection, transfection, gene transfer, microinjection, electroporation, transduction, and may be accomplished using, for example, a viral vector, a plasmid, or use of a gene gun. The methods of the invention may be carried out in vivo or ex vivo; ex vivo is preferred.

In another aspect of the invention, the capture of at least one apoptotic-cell-delivered antigen by a phagocyte may be enhanced by carrying out at least the steps of (a) expressing in a phagocytic cell an apoptotic cell receptor which will specifically direct the internalized apoptotic material in a manner facilitating the desired immunologic outcome, and (b) exposing the genetically-modified phagocyte to apoptotic cell(s) comprising an antigen(s). The apoptotic cells may comprise one or more antigens; alternately, a mixture of apoptotic cells each comprising at least one antigen, may be provided to the phagocytes. In a further embodiment, the phagocytic cell may be capable of cross-presenting the delivered antigen or antigens.

In another aspect of the invention, a genetically-modified phagocyte is provided, the phagocyte having the ability to capture, or an enhanced ability to capture, at least one apoptotic-cell-delivered antigen. The genetically-modified phagocyte is prepared by genetically modifying the phagocyte to express or increase expression of at least one apoptotic-cell receptor, or to increase its activity or function in capturing apoptotic cells, as described hereinabove. Thus, the cells may be modified to express or increase expression of at least one apoptotic-cell receptor, or by expression of at least one modified apoptotic cell receptor with enhanced affinity for apoptotic cells.

The invention is also directed to the various aforementioned integrin receptor β subunit modified polypeptides, including chimeras, as well as heterodimers comprising the modified β subunits, including chimeras, as well as polynucleotides encoding all of the preceding, as well as constructs, vectors, including any of the foregoing polypeptides or polynucleotides with labels such as green fluorescent protein (GFP), and other vehicles encoding or permitting expression of the various heretofore unknown modified integrin receptors as described throughout the specification.

In yet a further broad aspect, the present invention provides methods for enhancing the ability of a dendritic cell or a dendritic cell precursor to cross-present at least one apoptotic-cell-delivered antigen by genetically modifying the dendritic cell to increase its expression of at least one apoptotic-cell receptor or to express at least one apoptotic-cell receptor with enhanced ability to capture apoptotic cells. The dendritic cell may be a myeloid dendritic cell or a lymphoid dendritic cell. The at least one apoptotic-cell receptor may be, by way of non-limiting example, a member of the Fc receptor family, a member of the scavenger receptor family, a member of the ABC-1 family of transporters, a member of the C-type lectin family, an integrin receptor β subunit other than β₁, an integrin receptor heterodimer comprising a β subunit other than β₁, an integrin receptor heterodimer comprising a chimeric β subunit other than β₁, or an integrin heterodimer comprising a mutant β subunit, for example, a deletion or point mutation which provides a subunit with signaling properties similar to β₅. By way of example, the integrin receptor β subunit may be β₅, or the integrin receptor heterodimer may be αᵥβ₅. By way of further examples, the integrin heterodimer that comprises a chimeric β subunit may be a wildtype a subunit and a chimeric β subunit, wherein the chimeric β subunit is an extracellular β₅ domain fused with a signaling domain derived from a molecule such as an integrin receptor β subunit other than β₁, or derived from a member of the Fc receptor family, a member of the scavenger receptor family, or a member of the C-type lectin family. By way of further example, the signaling domain derived from a member of the Fc receptor family may be a FcRγI, FcRγIIA, FcRγIIB, or FcRγIII α-chain. The signaling domain derived from an integrin receptor β subunit other than β₁ may be that of β₂ or β₅.

Methods for genetically modifying the dendritic cell or dendritic cell precursor of this aspect of the invention are as described hereinabove.

In yet another broad aspect of the invention, a method is provided for enhancing the ability of a phagocyte other than a dendritic cell to capture and degrade at least one apoptotic-cell-delivered antigen by genetically modifying the phagocyte to express or increase the expression of at least one apoptotic-cell receptor or to express at least one apoptotic-cell receptor, including a modified apoptotic-cell receptor, with enhanced capture activity or function towards apoptotic cells. Non-limiting examples of phagocytes include professional phagocytes such as antigen presenting cells, an example thereof including macrophages. The phagocyte also may be a nonprofessional phagocyte, such as but not limited to a keratinocyte, a fibroblast, an epithelial cell or an endothelial cell. The phagocyte may be a human or a non-human phagocyte.

The apoptotic-cell receptor of the foregoing method may be, by way of non-limiting example, a member of the Fc receptor family, a member of the scavenger receptor family, CD14, a member of the ABC-1 family of transporters, a member of the C-type lectin family, an integrin receptor β subunit other than β₁, an integrin receptor heterodimer comprising a β subunit other than β₁, an integrin receptor heterodimer comprising a chimeric β subunit other than β₁, or an integrin receptor heterodimer comprising a mutant β subunit, as described herein. For example, the integrin receptor β subunit may be β₅; the integrin receptor heterodimer may be αᵥβ₅. The integrin receptor heterodimer comprising a chimeric β subunit may be a wild-type α subunit and a chimeric β subunit, wherein the chimeric β subunit is an extracellular β domain fused with a signaling domain derived from a molecule such as but not limited to an integrin receptor β subunit other than β₁, a member of the Fc receptor family, a member of the scavenger receptor family, or a member of the C-type lectin family. By way of example, the signaling domain derived from a member of the Fe receptor family may be a FcRγI, FcRγIIA, FcRγIIB or FcRγIII α-chain; the signaling domain derived from an integrin receptor β subunit other than β₁ may be that from β₂, β₃ or β₅.

Methods for genetically modifying the phagocyte of the foregoing method may be carried out by a method selected such as infection, transfection, microinjection, electroporation, or gene transfer, using such means as a viral vector, a plasmid, and use of a gene gun, as described above.

In a further aspect of the invention, a method is provided for enhancing the ability of a dendritic cell or a dendritic cell precursor to capture and degrade at least one apoptotic-cell-delivered antigen by genetically modifying the dendritic cell or dendritic cell precursor to express at least one apoptotic-cell receptor which is, for example, an integrin receptor heterodimer comprising an αᵥ subunit and a β₁ or β₃ subunit, or a chimeric β subunit with a β₁ or β₃ signaling domain, or to increase expression of an endogenous apoptotic-cell receptor. Genetic modification and other aspects of this embodiment are as described hereinabove.

In yet another aspect of the invention, a method is provided for enhancing the cross-priming of T cells by dendritic cells with at least one apoptotic-cell-delivered antigen by carrying out at least the steps of (a) genetically modifying the dendritic cells or precursors thereof to express or increase expression of at least one apoptotic-cell receptor capable of enhancing capture of apoptotic cells and trafficking internalized apoptotic material to achieve an immunological outcome which is cross-priming of T cells; and then (b) exposing the genetically-modified dendritic cells to at least one apoptotic cell comprising an antigen, in the presence of immunostimulatory exogenous factor(s) or antigen-specific CD4 helper T cells; wherein the dendritic cells have enhanced ability to promote the formation of antigen-specific CD8 cells. The immunostimulatory exogenous factor is at least one of CD40 ligand, TRANCE, TRAIL, OX40, or another a member of the TNF superfamily, or thalidomide. In accordance with this method, the apoptotic-cell receptor capable of promoting cross-priming of T cells may be an immunostimulatory member of the Fc receptor family (i.e., having an ITAM motif), a member of the scavenger receptor family, a member of the C-type lectin family, a β integrin receptor subunit other than β₁, an integrin receptor heterodimer other than that comprising β₁, an integrin receptor heterodimer comprising a chimeric β subunit other than β₁, or an integrin receptor heterodimer comprising a mutant β subunit. For example, the integrin receptor β subunit may be β₅, or the integrin receptor heterodimer may be αᵥβ₅. The integrin receptor heterodimer or β subunit may be a chimeric β subunit with an extracellular β₅ domain and an signaling domain such as but not limited to integrin β₂, integrin β₃, integrin β₅, or a FcγIR α-chain, FcγIIA α-chain or FcγRIII α-chain, or An immunostimulatory signaling sequence (ITAM) of the Fc γ-chain.

The dendritic cells may be myeloid dendritic cells or lymphoid myeloid dendritic cells. The apoptotic cell-delivered antigen may be, by way of non-limiting example, a tumor antigen and the T cells may be tumor-specific T cells. As noted herein, with regard to all of the embodiments of the invention, the apoptotic cells comprise at least one antigen, which may be for example expressed, carried, bound, or in any other manner be part of the apoptotic cells. Alternatively, a mixture of two or more populations of apoptotic cells, each comprising a different antigen, may be used to provide a plurality of antigens. In another embodiment, the antigen may be a viral antigen and the resulting enhanced T cells may be virus-specific or virally-infected-cell specific T cells. Any other CTL target antigens may also be used. The enhanced cross-priming of T cells with the antigen in accordance with the aforementioned method may be carried out to provide enhanced killing of tumors or virus-infected cells, among other activities directed at cell killing. Furthermore, the enhanced cross-priming of T cell may result in the enhanced formation of antigen-specific CD4 helper cells.

In yet a further aspect of the invention, a method is provided for enhancing the cross-tolerance of T cells to at least one apoptotic-cell-delivered antigen by dendritic cells or dendritic cell precursors by at least the steps of (a) genetically modifying the dendritic cells or precursors thereof to express or increase expression of at least one apoptotic-cell receptor capable of promoting capture of apoptotic cells and trafficking the internalized apoptotic material to enhance cross-tolerance of T cells; and (b) exposing the genetically-modified phagocytes to at least one apoptotic cell comprising an antigen, in the presence of at least one immunosuppressive exogenous factor or in the absence of the combination of antigen-specific CD4 helper T cells and at least one immunostimulatory exogenous factor; wherein the dendritic cells have increased ability tolerize antigen-specific CD8 cells. Non-limiting examples of immunosuppressive exogenous factors include TGF-β, IL-10, IL-4, IL-5, IL-13, FK506 (tacrolimus) or an agent that binds to FKBP12. The apoptotic-cell receptor which is capable of enhancing cross-tolerance of T cells may be an integrin receptor heterodimer with a β₂ subunit, a cross-tolerance inducing member of the FcR family, or a chimeric integrin receptor β subunit with an extracellular β₅ domain and an signaling domain such as integrin β₂ or FcγRIIB α-chain, or a Fc γ-chain with an immunosuppressive (ITIM) motif. In a further aspect of the invention, the cross-tolerance results in a decrease in autoreactive T cells to the antigen. The method described above may be used for treating, for example, an autoimmune disease, such as but not limited to psoriasis, Crohn's disease, rheumatoid arthritis, or multiple sclerosis. Furthermore, the method may be used prophylactically or therapeutically to reduce the immune response to and to tolerize CD8 cells to transplant antigen; wherein the antigen is, for example, one or more allogeneic transplant antigens or xenogeneic transplant antigens. The method may also result in tolerizing of CD4 helper cells to said antigen, or tolerizing of B cells to said antigen. Other prophylactic and therapeutic outcomes for eliciting tolerance to an antigen or a plurality of antigens may be achieved by these means.

In yet still a further aspect of the invention, a general method is provided for enhancing clearance (immune ignorance) to at least one apoptotic cell-delivered antigen. A method is provided for enhancing clearance directed to at least one apoptotic-cell-delivered antigen by a phagocyte other than a dendritic cell by at least the steps of (a) genetically modifying a phagocyte other than a dendritic cell to increase expression of at least one apoptotic-cell receptor capable of enhancing capture of apoptotic cells and promoting degradation of the antigen, or expressing at least one receptor with enhanced ability to capture apoptotic cells and promote degradation of antigen; and (b) introducing the genetically-modified phagocyte into diseased tissue of an individual. The genetic modification may be performed ex vivo, in vitro, or in vivo, including the use of phagocytes other than that of the individual, and subsequently introduced thereto. The apoptotic-cell receptor may be, by way of non-limiting example, a member of the Fc receptor family, a member of the scavenger receptor family, CD14, a member of the ABC-1 family of transporters, a member of the C-type lectin family, an integrin receptor β subunit other than β₁, an integrin receptor heterodimer comprising a β subunit other than β₁, an integrin receptor heterodimer comprising a chimeric β subunit other than β₁, or an integrin receptor heterodimer comprising a mutant β subunit. In one embodiment, the integrin receptor β subunit is β₅, or the integrin receptor heterodimer is αᵥβ₅. Alternately, the integrin receptor heterodimer comprising a chimeric β subunit may be a wild-type α subunit and a chimeric β subunit, wherein the chimeric β subunit is an extracellular β₅ domain fused with a signaling domain derived from a molecule such as an integrin β subunit other than β₁, a member of the Fc receptor family, a member of the scavenger receptor family, or a member of the C-type lectin family. The signaling domain derived from a member of the Fc receptor family may be a FcRγI, FcRγIIA, FcRγIIB or FcRγIII α-chain. The signaling domain derived from an integrin β subunit other than β₁ may be that of β₂, β₃ or β₅. Methods for genetically modifying the phagocyte are those described hereinabove. The method of this aspect if the invention may be used to enhance the clearance of apoptotic corpses in vivo, such as may be useful for the treatment of diseases such as lupus where a defect in apoptotic corpse clearance induces undesirable episodic immunologic reactions. Other conditions in which defective clearance of apoptotic cells is pathogenetic may be treated by these methods.

In a further aspect of the invention, a method is provided for enhancing cross-priming of T cells by dendritic cells or precursors thereof using an apoptotic-cell-delivered antigen by carrying out at least the steps of

(a) genetically modifying the dendritic cells or precursors thereof to increase expression of at least one integrin receptor heterodimer capable of capturing apoptotic cells or expressing at least one integrin receptor heterodimer with enhanced ability to capture apoptotic cells, wherein captured apoptotic material is trafficked to result in cross-priming, such as

i) αᵥβ₅;

ii) an integrin receptor heterodimer of αᵥ and a chimeric β subunit comprising an extracellular β₅ domain and a Fc FcγRI, FcγRIIA, or FcγIII α-chain signaling domain;

iii) a heterodimer of αᵥ and a chimeric β subunit comprising an extracellular β₅ domain and an integrin receptor β₃ or β₅ signaling domain;

iii) a β₅ subunit alone or a chimeric β subunit alone comprising an extracellular β₅ domain and an integrin β₃ or β₅ signaling domain; or

iv) a chimeric β subunit alone comprising an extracellular β₅ domain and a Fc FcγRI, FcγRIIA, or FcγRIII α-chain signaling domain;

(b) exposing the genetically-modified phagocyte to at least one apoptotic cell comprising at least one antigen in the presence of at least one immunostimulatory exogenous factor or antigen-specific CD4 helper T cells;

wherein the dendritic cells or precursors thereof have enhanced ability to form antigen-specific CD8 cells.

The foregoing method may be carried out in vitro, ex vivo, or in vivo; ex vivo is preferred. The immunostimulatory exogenous factor may be at least one of CD40 ligand, TRANCE, TRAIL, OX40, or another member of the TNF superfamily, or thalidomide; the member of the TNF superfamily may be TRAIL. In particular embodiments of the method, the antigen may be a tumor antigen and the T cells (CD8) are tumor specific T cells, or the antigen is a viral antigen and said T cells are virus-specific or virally-infected cell specific T cells. Other antigens or combinations may be used. The enhanced cross-priming of T cells with the antigen may result in enhanced killing of tumors or virus-infected cells. The dendritic cells may be lymphoid or mycloid dendritic cells. Other aspects of the method are as described hereinabove.

In still yet a further aspect of the invention, a method is provided for enhancing cross-tolerance to at least one apoptotic-cell-delivered antigen by dendritic cells or precursors thereof by carrying out at least the steps of

(a) genetically modifying the dendritic cells or precursors thereof to increase expression of at least one integrin heterodimer capable of capturing apoptotic cells or expressing at least one integrin heterodimer with enhanced ability to capture apoptotic cells, an trafficking the apoptotic material to achieve the immunologic outcome promoting cross-tolerance, such as

i) an integrin receptor heterodimer of αᵥ and a chimeric β subunit comprising an extracellular β₅ domain and a signaling β₂ domain;

ii) a chimeric β subunit alone comprising an extracellular β₅ domain and a signaling β₂ domain; or

iii) a chimeric β subunit alone comprising an extracellular β₅ domain and a signaling FcγRIIB domain;

(b) exposing the genetically-modified phagocyte to at least one apoptotic cell comprising at least one antigen in the presence of at least one immunosuppressive exogenous factor or in the absence of the combination of antigen-specific CD4 helper T cells and at least one immunostimulatory exogenous factor;

wherein the dendritic cells have reduced ability to cross-prime T cells with the antigen. The dendritic cells may then be introduced into the body.

In the practice of the foregoing method, the immunosuppressive exogenous factor may be, for example, TGF-β, IL-10, L-4, IL-5, or IL-13, FK506 (tacrolimus) or an agent that binds to FKBP12. The method may be used for treating an autoimmune disease, such as but not limited to psoriasis, Crohn's disease, rheumatoid arthritis, or multiple sclerosis. In addition, the method may be used for reducing the immune response to a transplant antigen, where the antigen is an allogeneic transplant antigen or a xenogeneic transplant antigen. Other immunosuppressive uses of the method towards one or more antigens are embraced herein.

In another preferred embodiment of the invention, a method is provided for stimulating the immune response in a mammalian patient to at least one preselected antigen to enhance the formation of antigen-specific CD8 cells comprising the steps of

a) obtaining a source of dendritic cells or precursors thereof;

b) genetically modifying the dendritic cells or precursors thereof with at least one apoptotic-cell receptor capable of promoting capture of apoptotic cells and enhancing cross-priming of an antigen, as described above;

c) exposing the genetically modified dendritic cells or precursors thereof to apoptotic cells expressing at least one antigen in the presence of at least one of the following compositions:

i) an agent capable of both facilitating cross-priming and maturing said dendritic cell; or

ii) the combination of at least one agent capable of facilitating cross-priming but not capable of maturing said dendritic cell, and at least one agent capable of inducing dendritic cell maturation but not capable of facilitating cross-priming;

d) optionally isolating the dendritic cells; and

e) administering the dendritic cells to a patient in need thereof.

The dendritic cell may be a myeloid dendritic cell or a lymphoid dendritic cell. The cells may be a non-human antigen presenting cell with features similar to a dendritic cell. The source of dendritic cells may be but is not limited to allogeneic cord blood, xenogeneic antigen presenting cells, bone marrow biopsy, bone marrow-derived dendritic cell precursors, isolated dendritic cell precursors, cells obtained by leukapheresis, dendritic cells mobilized from the bone marrow to the peripheral blood. An agent capable of both facilitating cross-priming and maturing the dendritic cells may be a member of the TNF superfamily (e.g., CD40 ligand, TRAIL, or OX40. An agent capable of facilitating cross-priming but not capable of maturing said phagocyte may be TRANCE or thalidomide or IL-12. An agent capable of inducing phagocyte maturation but not capable of facilitating cross-priming may be monocyte conditioned medium, L-6, TNF-α, IL-1β or PGE₂. These are merely non-limiting examples of suitable agents.

The apoptotic-cell receptor capable of promoting capture and cross-priming of T cells may be, by way of non-limiting example, a cross-priming promoting member of the Fc receptor family, a member of the scavenger receptor family, a member of the C-type lectin family, a β integrin receptor subunit other than β₁ or β₃, an integrin receptor heterodimer other than that comprising β₁ or β₃, an integrin receptor heterodimer comprising a chimeric β subunit other than β₁ or β₃, or an integrin receptor heterodimer comprising a mutant β subunit as described herein. For example, the integrin β subunit is β₅, or the integrin heterodimer may be αᵥβ₅. The integrin heterodimer or β subunit may be a chimeric β subunit with an extracellular β₅ domain and an signaling domain with the activity similar to that of integrin β₅, integrin β₂, FcγRI α-chain, FcγIIA α-chain or FcγRIII α-chain.

In the foregoing example, the antigen may be a tumor antigen and the T cells that are enhanced are tumor specific T cells; or the antigen may be a viral antigen and the enhanced T cells are virus-specific or virally-infected cell specific T cells. The enhanced cross-priming of T cells with said antigen by the foregoing method may result in enhanced killing of tumors or virus-infected cells. While the method is preferably carried out ex vivo, in vivo methods may be employed. Moreover, with non-human derived dendritic-type cells, certain aspects may be carried out in vitro prior to introduction of cells to the patient.

In still yet another aspect of the instant invention, provided herein is a method for suppressing the immune response in a mammalian patent to at least one preselected antigen comprising the steps of

a) obtaining a source of dendritic cells of precursors thereof;

b) genetically modifying the phagocytes with at least one apoptotic-cell receptor capable of promoting apoptotic cell capture, cross-presentation of an apoptotic cell-delivered antigen and promoting cross-tolerance of the antigen;

c) exposing the genetically-modified phagocytes to apoptotic cells expressing the antigen in presence of at least one immunosuppressive exogenous factor or in the absence of the combination of CD4 helper T cells and at least one immunostimulatory exogenous factor;

d) optionally isolating the dendritic cells; and

e) administering the dendritic cells to a patient in need thereof.

The dendritic cells may be myeloid dendritic cells or lymphoid dendritic cells. The source of dendritic cells or precursors thereof may be, for example, allogeneic cord blood, xenogeneic antigen presenting cells, bone marrow biopsy, bone marrow-derived dendritic cell precursors, isolated dendritic cell precursors, cells obtained by leukapheresis. The dendritic cells may also be non-human cells with the properties of dendritic cells and capable of being introduced into a human to enhance immune suppression to the antigen.

The immunosuppressive exogenous factor may be TGF-β, IL-10, IL-4, IL-5, IL-13, FK506 (tacrolimus) or an agent that binds to FKBP12. The apoptotic-cell receptor capable of enhancing cross-tolerance of T cells may be an integrin heterodimer with a β₂ subunit or a chimeric β subunit with an extracellular β₅ domain and an signaling domain from integrin β₂ or the FcγIIB α-chain. The aforementioned method may be used for the treatment of an autoimmune disease, such as psoriasis, Crohn's disease, rheumatoid arthritis, or multiple sclerosis. It also may be used to tolerize T cells to a transplant antigen such as an allogeneic transplant antigen or a xenogeneic transplant antigen.

In another aspect of the invention, a method is provided for increasing the expression of an αβ integrin receptor heterodimer in a phagocyte comprising genetically modifying the phagocyte with only the integrin receptor β subunit, whether native, or a chimeric or mutant form thereof. Increased expression of the introduced β subunit has the effect of increasing expression of the endogenous a subunit and the enhanced appearance of heterodimers. In all of the foregoing embodiments, a β subunit alone with the desired properties of the heterodimer may be introduced into the phagocyte by genetic modification as embraced herein, to achieve the expression of an integrin receptor heterodimer with the desired β subunit and the α unit recruited thereby.

In another aspect of the invention, a method is provided of identifying means for altering processing of apoptotic cell-delivered antigens by a phagocytic cell comprising utilizing a 293T cell as a model phagocytic cell for such studies. Such cells and others with a dendritic receptor profile are useful in screens for modulators of dendritic cell activity.

It is thus a general object of the present invention to provide methods for modulating the immune response to at least one preselected antigen delivered to phagocytes by apoptotic cells, such that the immune response provides an enhanced cytotoxic T cell response, suppresses the T cell response to the antigen, or results in degradation and clearance of the antigen.

It is a further object to provide genetically-modified phagocytes, such as a dendritic cells, with enhanced ability to cross-present antigen, for the purpose or either enhancing or suppressing the immune response to the antigen. It is another object to provide phagocytic cells other than dendritic cells with enhanced ability to capture and degrade apoptotic cells.

These and other aspects of the present invention will be better appreciated by reference to the following drawings and Detailed Description.

### BRIEF DESCRIPTION OF THE DRAWINGS

FIGS. 1A-1E show that 293T cells efficiently phagocytose apoptotic cells.

FIGS. 2A-2D show that 293T cells capture apoptotic cells in a manner that is similar to immature dendritic cells.

FIGS. 3A-3B shows how bicistronic vectors are used to correlate green fluorescent protein (GFP) levels with expression of the integrin receptor β₅.

FIGS. 4A-4D show that β₅ integrin expression regulates phagocytosis of apoptotic cells.

FIGS. 5A-5D show that β₅ activation leads to recruitment of the p130cas/CrkII/DOCK180 molecular complex.

FIG. 6 shows that CrkII is critical for the phagocytosis of apoptotic cells.

FIGS. 7A-7C shows that c-CrkII localizes to the phagosome of the immature DC upon internalization of an apoptotic cell.

FIG. 8 shows that adenoviral infection of DCs does not alter maturation state nor β₅ expression.

FIG. 9 shows the kinetics of phagocytosis in 293T cells.

FIGS. 10A and 10B shows show that β₅ activation leads to recruitment of the p130cas/CrkII/DOCK180/Rac-1 molecular complex. Panel 10B is a control experiment using β₁.

### DETAILED DESCRIPTION OF THE INVENTION

The present invention is broadly directed to methods for enhancing the ability of the immune system to increase or stimulate, or decrease or suppress the normal cellular immune response to an antigen, for the purpose of either enhancing effectiveness of, for example, anti-viral and anti-tumor responses, decreasing immunological reactions in, for example, autoimmune disease or organ rejection; or, in another embodiment, simply clearing certain antigens (i.e., T-cell ignorance) to which an immunologic reaction is responsible for disease. These methods are based upon the previous discovery by certain of the inventors herein of the utility of the delivery of antigen to antigen presenting cells (APCs) by means of apoptotic cells. International application PCT/US99/03763 (WO 99/42564) describes this phenomenon in detail, and is incorporated herein by reference in its entirety. The present inventors have improved upon and expanded the aforementioned invention. Genetically manipulating or altering the phenotype of phagocytes to express apoptotic cell receptors was found to enhance their ability to capture apoptotic cells; furthermore, depending on the nature of the particular receptor, trafficking the apoptotic material to achieve certain immunological outcomes could be provided. Under certain circumstances, cross-presentation of apoptotic cell-delivered antigens occurs, and thus the immune response to the particular antigen can be altered, and more specifically, tailored for the treatment or prophylaxis of various diseases or conditions. Moreover, the enhanced cross-presentation can be exploited to increase or decrease the immune response to the antigen by way of either enhanced cross-priming, or alternately, enhanced cross-tolerance, respectively. As will be described in more detail below, at least three general immunologic outcomes may be obtained by particular manipulation of the immune system in accordance with the teachings herein, dependent on the type of phagocyte, the particular apoptotic cell receptor or features thereof, the microenvironment in which the preparation of the immune cells is performed, as well as other factors. These procedures may be performed in vivo, or preferably, ex vivo with immune cells from the patient or from another source, for later introduction or reintroduction into the patient, depending on the source(s) of the cells. The general outcomes are to either to 1) enhance the development of cytotoxic T cells (CD8 cells; CTLs) reactive with a particular antigen or antigens, for example, for enhanced recognition and killing of tumor cells, virally-infected cells, or other CTL targets for various infectious and noninfectious diseases; 2) suppressing the development of or tolerizing CD8 cells such that a reduced immune response to a particular antigen or antigens is achieved, for the purpose of decreasing the intensity of an immunologic reaction to an autoimmune antigen, or the response to an existing antigen or anticipated exposure to a transplant of foreign antigens; and 3) providing enhanced clearance of apoptotic cells in tissues and other regions of the body in conditions in which impaired clearance of apoptotic cells leads to pathology, such as in systemic lupus erythematosus. The foregoing examples of conditions and diseases are merely illustrative of a wide range of utilities to which the instant methods may be applied for the benefit of patients in general, whether humans or non-human mammals. The following detailed descriptions of these various aspects of the instant methods provide additional examples of the utilities of the invention. The skilled artisan will be aware of numerous variations that may be taken from the teaching herein to apply the instant invention to a wide variety of therapeutic and prophylactic uses; the present invention is not so limiting and embraces such other uses.

Before describing the various aspects of the invention in greater detail, for the purpose of a more complete understanding of the invention, the following definitions and general methods are described herein:

The term “apoptosis” means non-necrotic, energy-dependent cell death, which can occur under a variety of conditions including programmed cell death, exposure to ionizing and UV irradiation, serum starvation, activation of Fas and other tumor necrosis factor receptor-related pathways, and by drugs such as dexamethasone or an alternative steroid which induces apoptotic death; ceremide chemotherapeutic agents which trigger apoptotic death (e.g. Adriamycin); and antihormonal agents (e.g. Lupron, Tamoxofen). Apoptosis is characterized by, inter alia, formation of “blebs” and vesicles at the plasma membrane, cell shrinkage, pyknosis, and increased endonuclease activity (24, 25). Specific markers for apoptosis include, but are not limited to, annexin V staining, propidium iodide staining, DNA laddering, staining with dUTP and terminal transferase (TUNEL). The present invention is not so limited to any particular means for inducing apoptosis in a cell delivering one or more antigens for the intended purposes.

The term “apoptotic-cell-delivered antigen” or etymological variants thereof means any cell containing one or more native or foreign antigens undergoing apoptosis due to any condition, including those which were previously though to be associated with causing necrosis, but are now know to be on the spectrum of apoptotic death as ATP is required (e.g. complement mediated lysis). Thus, an apoptotic cell is identified based on its characteristics described above rather than any method used leading to cell death. Similarly, the term “apoplotic cell fragments” means apoptotic cell material, bodies, blebs, vesicles, or particles other than whole apoptotic cells which contain antigen. Such fragments are included in the meaning of apoptotic cells or apoptotic cell-delivered antigens for the purposes herein. The apoptotic cells or fragments of the invention may carry one or more antigens without any manipulation except optionally by isolation from the source or donor; otherwise, cells can be manipulated to contain an antigen by any of several means including but not limited to infection, transfection, or other forms of genetic manipulation in which the antigen is introduced into and expressed by the cell; loading the cell with the antigen(s); cross-linking antigens to the cell surface, use of cells expressing or containing the antigen without any manipulation, i.e., cells from another individual, etc. Furthermore, for the purposes herein, an apoptotic cell-delivered antigen may be one or more antigens, and the methods carried out either by use of an apoptotic cell population which contains more than one antigen, or by use of a mixture of two or more populations of apoptotic cells, each population of which contains a particular antigen.

The term “necrosis” means a form of energy-independent cell death resulting from irreversible trauma to cells typically caused by osmotic shock or exposure to chemical poison, and is characterized by marked swelling of the mitochondria and cytoplasm, followed by cell destruction and autolysis (26).

The term “donor cell” means the apoptotic cell that delivers antigen to dendritic cells for processing and presentation to T cells.

The phenomenon of “cross-priming” occurs when antigens from donor cells are acquired by the host APCs such as dendritic cells and are processed and presented on MHC molecules at the surface of the APC for activation of antigen-specific T cells.

The phenomenon of “cross-tolerance” occurs when antigens from donor cells are acquired by host dendritic cells and are presented under conditions that are non-inflammatory (lack of inflammation or other maturation stimuli) so as to cause antigen-specific unresponsiveness in T cells.

The term “antigen” means all, or parts thereof, of a protein, peptide, or other molecule or macromolecule capable of causing an immune response in a vertebrate preferably a mammal. Such antigens are also reactive with antibodies from animals immunized with said protein or other macromolecule. The potent accessory function of dendritic cells provides for an antigen presentation system for virtually any antigenic epitope which T lymphocytes are capable of recognizing through their specific receptors. As noted herein, the various aspects of the invention are intended to include one or more antigens, whether multiple antigens are contained with a certain population of apoptotic cells, or whether multiple apoptotic cells each expressing different antigens are mixed for use in the methods herein.

The term “genetically modified,” “genetically modifying,” “genetic manipulation,” and other syntactic variants and terms related thereto refer generally to the alteration of the genotype or phenotype of a cell by introduction into that cell from an exogenous source or alteration of the cell using an exogenous method, of a gene, genes, fragments of genes, mutations in existing genes, etc. Such methods and related terms include transfection, infection, transformation, transduction, etc. Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990). For example, a cell has been “transfected” by exogenous or heterologous DNA when such DNA has been introduced inside the cell. A cell has been “transformed” by exogenous or heterologous DNA when the transfected DNA effects a phenotypic change.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

“Heterologous” DNA refers to DNA not naturally located in the cell, or in a chromosomal site of the cell. Preferably, the heterologous DNA includes a gene foreign to the cell.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background.

Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667).

Accordingly, the term “sequence similarity” in all its grammatical forms refers to the degree of identity or correspondence between nucleic acid or amino acid sequences of proteins that do not share a common evolutionary origin (see Reeck et al., supra). However, in common usage and in the instant application, the term “homologous,” when modified with an adverb such as “highly,” may refer to sequence similarity and not a common evolutionary origin.

In a specific embodiment, two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Similarly, in a particular embodiment, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program.

The term “corresponding to” is used herein to refer similar or homologous sequences, whether the exact position is identical or different from the molecule to which the similarity or homology is measured. Thus, the term “corresponding to” refers to the sequence similarity, and not the numbering of the amino acid residues or nucleotide bases. A “chimera” as used herein refers to a β integrin receptor subunit which comprises an extracellular domain derived from one source, and a signaling domain from another. For example, for conferring or enhancing apoptotic cell capture, a β subunit chimera comprising an extracellular β₅ domain fused with a signaling domain derived from a molecule such as an integrin β subunit other than β₁, a member of the Fc receptor family, a member of the scavenger receptor family, or a member of the C-type lectin family. By way of example, the signaling domain derived from a member of the Fc receptor family may be the FcγRI, FcγRIIA, FcγRIIB or FcγRIII α-chain or any signaling sequence of a Fc γ-chain; the signaling domain derived from an integrin receptor β subunit other than β₁ may be that of β₂, β₃ or β₅. The extracellular domain may be another domain with the same properties as that of β₅. For cross-presentation, an extracelluar β₅ domain (or another with like properties) may be fused with a signaling domain derived from a molecule such as an integrin receptor β subunit other than β₁, or derived from a member of the Fc receptor family, a member of the scavenger receptor family, or a member of the C-type lectin family. By way of further example, the signaling domain derived from a member of the Fe receptor family may be a FcRγI, FcRγIIA, FcRγIIB, or FcRγIII α-chain. The signaling domain derived from an integrin receptor β subunit other than β₁ may be β₂ or β₅. For cross-priming, the chimera may comprise a chimeric β subunit with an extracellular β₅ domain and an signaling domain such as that from integrin receptor β₂, integrin β₃, integrin β₅, or a FcγRI α-chain, FcγIIA α-chain or FcγRIII α-chain, or an alternate immunostimulatory FcR γ-chain, i.e., one that includes an ITAM motif . For cross-tolerance, the chimera may be an extracellular β₅ domain (or other with like properties) and an signaling domain such as integrin receptor β₂ or FcγRIIB α-chain, or a Fc γ-chain with an immunosuppressive (ITIM) motif The integrin receptor β subunit of the invention may be a native form, or a mutant or chimeric form, the latter non-native forms referred to as “modified” in certain contexts herein. Integrin and integrin receptor are used interchangeably herein.

A “heterodimer” refers to an integrin receptor comprising an a subunit and a β subunit.

The following general methodologies are employed in the practice of the present invention.

General Molecular Techniques.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

For example, an identified and isolated gene can be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2μ plasmid.

In an alternative method, the desired gene may be identified and isolated after insertion into a suitable cloning vector in a “shot gun” approach. Enrichment for the desired gene, for example, by size fractionation, can be done before insertion into the cloning vector.

Sources of Dendritic Cells

The dendritic cells used in this invention can be isolated as described herein or by methods known to those skilled in the art. In a preferred embodiment, human dendritic cells are used from an appropriate tissue source, preferably cord blood, peripheral blood or bone marrow.

Mature dendritic cells can also be obtained by culturing proliferating or non-proliferating dendritic cell precursors in a culture medium containing factors which promote maturation of immature dendritic cells to mature dendritic cells. Steinman et al. U.S. Pat. No. 5,851,756 and U.S. application Ser. No. 08/600,483 and WO 97/29182 report methods and compositions for obtaining dendritic cells and are incorporated herein by reference.

The dendritic cell precursors, from which the immature dendritic cells for use in this invention are derived, are present in blood as PBMCs. Although most easily obtainable from blood, the precursor cells may also be obtained from any tissue in which they reside, including cord blood, bone marrow and spleen tissue. When cultured in the presence of cytokines such as a combination of GM-CSF and IL-4 or IL-13 as described below, the non-proliferating precursor cells give rise to immature dendritic cells for use in this invention. In the present invention, a preferred embodiment is isolation of dendritic cells from whole blood.

Culture of Pluripotential PMBCs to Produce Immature Dendritic Cells

Dendritic cell development can be divided into 4 stages: 1) a proliferating progenitor that can be either dendritic cell committed or uncommitted and capable of maturing to a nondendritic cell, 2) a non-proliferating precursor like the blood monocyte that does not show dendritic cell properties but is the starting population for many clinical studies, 3) an immature dendritic cell which has properties and commitment to become a dendritic cell, e.g. specialized antigen capture mechanisms including apoptotic cells for presentation, and MHC rich compartments, and 4) finally, the mature T cell stimulatory dendritic cell, also referred to as “superactivated,” which is capable of cross-priming T cells.

Cultures of immature dendritic cells, i.e. antigen-capturing phagocytic dendritic cells, may be obtained by culturing the non-proliferating precursor cells in the presence of cytokines which promote their differentiation. A combination of GM-CSF and IL-4 at a concentration of each at between about 200 to about 2000 U/ml, more preferably between about 500 and 1000 U/ml, and most preferably about 800 U/ml (GM-CSF) and 1000 U/ml (IL-4) produces significant quantities of the immature, i.e. antigen-capturing phagocytic dendritic cells, dendritic cells. Other cytokines or methods known in the art which efficiently generate immature dendritic cells may be used for purposes of this invention. Other cytokines which promote differentiation of precursor cells into immature dendritic cells include, for example, IL-13. Maturation of dendritic cells requires the addition to the cell environment, preferably the culture medium, of a dendritic cell maturation factor which may be selected from monocyte conditioned medium and/or factors including TNF-α, IL-6, IFN-α, and IL-1β. Alternatively, a mixture of necrotic cells or necrotic cell lysate may be added to induce maturation. In the present invention, a preferred embodiment is isolation of dendritic cells from peripheral blood.

Co-Culture of Dendritic Cells with Apoptotic Cells

Apoptotic cells may be used to deliver antigen to either immature or mature dendritic cells, either freshly isolated or obtained from in-vitro culture. In a preferred embodiment, apoptotic cells comprising an antigen are co-cultured with immature dendritic cells, genetically modified as described herein, for a time sufficient to allow the antigen to be internalized by the immature dendritic cells. As noted above, the dendritic cells comprising antigen may be obtained or prepared to contain and/or express one or more preselected antigens by any of a number of means, such that the antigen(s) is (are) delivered to the phagocyte upon capture of the apoptotic cell. These immature dendritic cells are then caused to mature by the addition of a maturation factor to the culture medium. The matured dendritic cells expressing processed antigen on their surfaces are then exposed to T cells for potent CTL induction. As noted herein, the genetic modification enhances the capture of the apoptotic cells by dendritic cells, and further, directs the internalized apoptotic material to the desired immunological outcome, such as cross-priming, cross-tolerance, or degradation and clearance (immune ignorance).

For example, in one embodiment, peripheral blood mononuclear cells (PBMCs) are isolated from blood by sedimentation techniques. T cell-enriched (ER⁺) and T cell-depleted (ER⁻) populations are prepared by rosetting with neuraminidase-treated sheep red blood cells. Dendritic cells are prepared from the ER⁻ cells (Steinman et al., application Ser. No. 08/600,483, incorporated herein by reference in its entirety) as discussed above and are preferably cultured for 5 days to 8 days in the presence of GM-CSF and IL-4. On about day 7 through 10, apoptotic cells can be co-cultured with the dendritic cells and the dendritic cells caused to mature over the next two to four days with the addition of monocyte conditioned medium, a signal for maturation.

Besides monocyte conditioned medium, a combination of cytokines may be used to induce maturation of the immature dendritic cells. Examples of cytokines which may be used alone or in combination with each other include, but are not limited to, TNFα, IL-1β, IL-6, IFNα and necrotic cells.

The apoptotic cell-activated dendritic cells made according to the method described above are the most efficient for induction of CTL responses. Delivery of antigen to mature dendritic cells, or alternatively, immature dendritic cells that are not caused to mature in vitro, is also within the scope of this invention.

The apoptotic cells useful for practicing the method of this invention should efficiently trigger antigen internalization by dendritic cells, and once internalized, facilitate translocation of the antigen to the appropriate antigen processing compartment.

In a preferred embodiment, the apoptotic cells, or fragments, blebs or bodies thereof, are internalized by the dendritic cells and targeted to an MHC class I processing compartment for activation of class I-restricted CD8⁺ cytotoxic T cells.

In another embodiment, the apoptotic cells can be used to activate class II-restricted CD4⁺ T helper cells by targeting antigen via the exogenous pathway and charging MHC class II molecules. Apoptotic cells, blebs and bodies are acquired by dendritic cells by phagocytosis. When a population of CD4⁺ cells is co-cultured with apoptotic cell-primed dendritic cells, the CD4+ T cells are activated by dendritic cells that have charged their MHC class II molecules with antigenic peptides. The apoptotic cell-charged dendritic cells of this invention activate antigen-specific CD4+ T cells with high efficiency.

For purposes of this invention, any cell type which contains antigen and is capable of undergoing apoptosis can potentially serve as a donor cell for antigen delivery to the potent dendritic cell system. These include whole cells which are themselves the antigen(s) for which a modified immune response is desired, such as virally-infected cells, bacterial cells, protozoan cells, microbial cells and tumor cells expressing tumor antigens, as well as self-antigens. Such particular antigens may also be introduced into other cells types which may then be made apoptotic for delivery to the phagocytes in accordance with the invention. Preferred antigens for priming dendritic cells in vitro or in vivo are derived from influenza virus, malaria, HIV, EBV human papilloma virus (including both EBV-associated and EBV-unassociated lymphomas), CMV, renal cell carcinoma antigens, and melanoma antigens. Other cancers with antigens of interest include prostate and breast, but the invention is not so limiting and embraces all dysproliferative diseases. In addition, self antigens that are targets of autoimmune responses can be delivered to dendritic cells e.g. insulin, histones, and GAD65.

For purposes of this invention the population of donor cells containing antigen can be induced to undergo apoptosis in vitro, including ex vivo, or in vivo using a variety of methods known in the art including, but not limited to, viral infection, irradiation with ultraviolet light, gamma radiation, cytokines or by depriving donor cells of nutrients in the cell culture medium. Time course studies can establish incubation periods sufficient for optimal induction of apoptosis in a population of donor cells. For example, monocytes infected with influenza virus begin to express early markers for apoptosis by 6 hours after infection. Examples of specific markers for apoptosis include annexin V, TUNEL+ cells, DNA laddering and uptake of propidium iodide.

Those skilled in the art will recognize that optimal timing for apoptosis will vary depending on the donor cells and the technique employed for inducing apoptosis. Cell death can be assayed by a variety of methods known in the art including, but not limited to, fluorescence staining of early markers for apoptosis, and determination of percent apoptotic cells by standard cell sorting techniques.

In one embodiment, donor cells are induced to undergo apoptosis by irradiation with ultraviolet light. Depending on the cell type, typically exposure to UV light (60 mjoules/cm²/sec) for 1 to 10 minutes induces apoptosis. This technique can be applied to any cell type, and may be most suitable for a wide range of therapeutic applications. The apoptotic donor cells containing an antigen of interest could then be used to prime dendritic cells in vitro or in vivo.

In another embodiment, donor cells are induced to undergo apoptosis by use of a drug such as dexamethasone or an alternative steroid which induces apoptotic death; ceremide chemotherapeutic agents which trigger apoptotic death (e.g. Adriamycin); and anti-hormonal agents (e.g. Lupron, Tamoxofen) which induces apoptosis. This technique can also be applied to any cell type, and is also suitable for a wide range of therapeutic applications.

In another embodiment, donor cells are induced to undergo apoptosis by infection with influenza virus. These apoptotic cells which express viral antigens on their surface could then be used to prime dendritic cells in vitro or in vivo. The apoptotic cell-activated dendritic cells may then be used to activate potent influenza-specific T cells.

In another embodiment, tumor cells may be obtained and caused to undergo apoptosis. These apoptotic tumor cells, or tumor cell lines, could then be used to deliver tumor antigen to dendritic cells in vitro or in vivo. Once isolated, the tumor cells could be treated with collagenase or other enzymes which facilitate cell dissociation for culturing. The apoptotic cell-activated dendritic cells may then be used as cancer therapeutic agents by activating the immune system to specifically target the tumor cells.

In another embodiment of the invention, the donor cells can be infected, transfected, transduced or transformed to express foreign antigens prior to induction of apoptosis. The cells may also be, for example, osmotically loaded or infected with bacteria containing a foreign antigen, prior to induction of apoptosis. In this manner dendritic cells may be loaded with antigens not typically expressed on the donor cell. In addition, delivery of antigens via xenotransfer is also contemplated. These methods can be accomplished using standard techniques known in the art. As noted above, more than one preselected antigen can be provided for modulation of the immune response as described herein.

A variety of possible antigens can be used in this invention including, but not limited to, bacterial, parasitic, fungal, viral, and tumor antigens of cellular or viral origin. Preferred antigens include influenza virus, malaria, HIV, EBV, human papilloma virus, CMV, renal cell carcinoma antigens, and melanoma antigens. In addition, self antigens that are targets of autoimmune responses or other antigens for which it is desired to attenuate an immune response can be expressed on donor cells using any of the aforementioned methods. Examples of self-antigens include, but are not limited to, lupus autoantigen, Ro, La, U1-RNP, Smith antigen (scleroderma), GAD65 (diabetes-related), myelin basic protein, PLP, collagen, etc.

Once donor cells expressing at least one native or foreign antigen, or a combination thereof, have been induced to undergo apoptosis, they can be contacted with an appropriate number of dendritic cells in vitro or in vivo. The ratio of apoptotic cells to dendritic cells may be determined based on the methods disclosed in herein and in prior studies, adjusted for the enhanced capture of apoptotic cells by the genetic modification using the apoptotic cell receptors of the invention. For most antigens a ratio of only about 1-10 donor cells to 100 dendritic cells is suitable for priming the dendritic cells.

The population of apoptotic cells should be exposed to the dendritic cells for a period of time sufficient for the dendritic cells to internalize the apoptotic cell, or apoptotic cell fragments. Efficiency of cross-priming or cross-tolerizing dendritic cells can be determined by assaying T cell cytolytic activity in vitro or using dendritic cells as targets of CTLs. Other methods known to those skilled in the art may be used to detect the presence of antigen on the dendritic cell surface following their exposure to apoptotic donor cells. Moreover, those skilled in the art will recognize that the length of time necessary for an antigen presenting cell to phagocytose apoptotic cells, or cell fragments, may vary depending on the cell types and antigens used, as well as the type of receptor genetically introduced, in accordance with the teachings herein.

An important feature of the dendritic cells of this invention is the capacity to efficiently present antigens on both MHC class I and class II molecules. Apoptotic donor cells, blebs, bodies or fragments thereof, are acquired by dendritic cells through the exogenous pathway by phagocytosis and as a result also efficiently charge MHC II molecules. CD4+ T cells may be activated by the dendritic cells presenting antigenic peptide which is complexed with MHC II using the method according to this invention, since it is known in the art that dendritic cells are the most potent inducers of CD4+ helper T cell immunity. CD4+ T cells can provide critical sources of help, both for generating active CD8+ and other killer T cells during the acute response to antigen, and for generating the memory that is required for long term resistance and vaccination. Thus, by using apoptotic cells to charge MHC class I and/or II products, efficient T cell modulation in situ can be achieved.

Dendritic cells, as modified by the procedures herein, may be administered to an individual using standard methods including intravenous, intraperitoneal, subcutaneously, intradermally or intramuscularly. The homing ability of the dendritic cells facilitates their ability to find T cells and cause their activation.

By adapting the system described herein, dendritic cells as modified herein could also be used for generating large numbers of CD8⁺ CTL, for adoptive transfer to immunosuppressed individuals who are unable to mount normal immune responses. Immunotherapy with CD8⁺ CTL has been shown to amplify the immune response. Bone marrow transplant recipients given CMV specific CTL by adoptive transfer, do not develop disease or viremia (30). These novel approaches for vaccine design and prophylaxis should be applicable to several situations where CD8⁺ CTLs are believed to play a therapeutic role e.g. HIV infection (30-32), malaria (33) and malignancies such as but not limited to melanoma (27, 28).

Examples of diseases that may be treated by the methods disclosed herein include, but are not limited to bacterial infections, protozoan, such as malaria, listeriosis, microbial infections, viral infections such as HIV or influenza, cancers or malignancies such as melanoma, autoimmune diseases such as psoriasis and ankolysing spondylitis.

Expression of Apoptotic Cell Receptors

The polynucleotide sequence coding for an apoptotic-cell receptor, or antigenic fragment, derivative or analog thereof, or a functionally active derivative, including a chimeric protein, thereof, can be inserted into an appropriate expression vector, i.e., a vector which contains the necessary elements for the transcription and translation of the inserted protein-coding sequence. Such elements are termed herein a “promoter.” Thus, the nucleic acid encoding the apoptotic-cell receptor of the invention is operationally associated with a promoter in an expression vector of the invention. Both cDNA and genomic sequences can be cloned and expressed under control of such regulatory sequences. An expression vector also preferably includes a replication origin.

The necessary transcriptional and translational signals can be provided on a recombinant expression vector, or they may be supplied by the native gene encoding apoptotic-cell receptor and/or its flanking regions.

Potential host-vector systems include but are not limited to mammalian cell systems infected with virus (e.g., vaccinia virus, adenovirus, lentivirus, pseudotype viruses, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Non-limiting examples of such means for expression are described in the Examples below.

Gene Therapy and Transgenic Vectors

In one embodiment, a gene encoding an apoptotic-cell receptor protein or polypeptide domain fragment thereof is introduced in vitro, in vivo or ex vivo using a viral vector. Such vectors include an attenuated or defective DNA virus, such as but not limited to herpes simplex virus (HSV), papillomavirus, Epstein Barr virus (EBV), adenovirus, adeno-associated virus (AAV), pseudotype virus and the like. Defective viruses, which entirely or almost entirely lack viral genes, are preferred. Defective virus is not replicative after introduction into a cell. For in-vivo among other embodiments of the invention, use of defective viral vectors allows for administration to cells in a specific, localized area, without concern that the vector can infect other cells. Thus, a desired tissue can be specifically targeted. Examples of particular vectors include, but are not limited to, a defective herpes virus 1 (HSV1) vector [Kaplitt et al., Molec. Cell. Neurosci. 2:320-330 (1991)], an attenuated adenovirus vector, such as the vector described by Stratford-Perricaudet et al. [J. Clin. Invest. 90:626-630 (1992)], and a defective adeno-associated virus vector [Samulski et al., J. Virol. 61:3096-3101 (1987); Samulski et al., J. Virol. 63:3822-3828 (1989)].

In another embodiment the gene can be introduced in a retroviral vector, e.g., as described in Anderson et al., U.S. Pat. No. 5,399,346; Mann et al., 1983, Cell 33:153; Temin et al., U.S. Pat. No. 4,650,764; Temin et al., U.S. Pat. No. 4,980,289; Markowitz et al., 1988, J. Virol. 62:1120; Temin et al., U.S. Pat. No. 5,124,263; International Patent Publication No. WO 95/07358, published Mar. 16, 1995, by Dougherty et al.; and Kuo et al., 1993, Blood 82:845.

Targeted gene delivery is described in International Patent Publication WO 95/28494, published October 1995.

Alternatively, the vector can be introduced in vivo by lipofection, the use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker [Felgner, et. al., Proc. Natl. Acad. Sci. U.S.A. 84:7413-7417 (1987); see Mackey, et al., Proc. Natl. Acad. Sci. U.S.A. 85:8027-8031 (1988)]. The use of cationic lipids may promote encapsulation of negatively charged nucleic acids, and also promote fusion with negatively charged cell membranes [Felgner and Ringold, Science 337:387-388 (1989)]. The use of lipofection to introduce exogenous genes into the specific organs in vivo has certain practical advantages. Molecular targeting of liposomes to specific cells represents one area of benefit.

It is also possible to introduce the vector in vivo or ex vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter [see, e.g., Wu et al., J. Biol. Chem. 267:963-967 (1992); Wu and Wu, J. Biol. Chem. 263:1462114624 (1988); Hartmut et al., Canadian Patent Application No. 2,012,311, filed Mar. 15, 1990].

In a preferred embodiment of the present invention, a gene therapy vector as described above employs a transcription control sequence operably associated with the sequence for the apoptotic-cell receptor inserted in the vector. That is, a specific expression vector of the present invention can be used in gene therapy.

As noted above, three general areas of utility comprise the present invention. These are described in more detail below.

### Enhanced Capture of Apoptotic Cell-Delivered Antigens by Phagocytes

As described above, one general aspect of the present invention is the genetic modification of phagocytes to enhance capture of apoptotic cells. In one embodiment, the genetic modification is provided to cause expression of receptors capable of recognizing and engulfing apoptotic cells, and in particular, apoptotic cells containing a preselected antigen for which modulation of the immune response thereto is desired. In another embodiment, the genetic modification provides expression of an apoptotic cell receptor with enhanced ability to capture apoptotic cells. The recognition and engulfment of apoptotic cells is referred to herein as capture. While the subsequent and desired effects of the enhanced capture are described in later sections below (e.g., enhanced clearance, cross-presentation and cross-priming, or cross-presentation and cross-tolerance), as provided by the nature of the receptor and the trafficking of the internalized apoptotic material, this aspect of the invention is applicable to all of the desired outcomes herein. Thus, the discussion herein is general to the types of cells modified, the types of receptors, and the like, and is not dependent on any particular desired subsequent outcome.

The methods herein are generally applicable to phagocytes, cell capable of capturing apoptotic cells. In particular embodiments, the phagocytes for which the methods herein are applicable include both professional phagocytes and non-professional phagocytes. Examples of professional phagocytes include antigen presenting cells, which include dendritic cells, macrophages, B cells, and neutrophils, to name some non-limiting examples. Dendritic cells may be myeloid dendritic cells or a lymphoid dendritic cells. The invention also embraces nonprofessional phagocytes, such as keratinocytes, epithelial cells, fibroblasts, or endothelial cells. The methods may be applied to human phagocytes or non-human phagocytes. For the purposes of development of screening methods to identify receptors and other parameters to enhance phagocytic cell capture, and as it will be seen in more detail below, 293T cells and other cells having a similar receptor profile with dendritic cells thus share properties with dendritic cells are useful for in-vitro studies of enhanced phagocytic cell capture of apoptotic cells. Moreover, the aforementioned cells may be from any animal species, preferably mammal and most preferably human, although for certain purposes which will be elaborated on below, phagocytic cells from insect or other non-human or even non-mammalian species may be used for the practice of the invention, particularly for the cross-priming of T cells with an apoptotic cell-delivered antigen.

Although the enhanced capture by increasing expression of an apoptotic-cell receptor is a general aspect of the instant invention, as it will be noted below, certain of the aforementioned cell types will be more applicable to certain desired outcomes than others; for example, dendritic cells or dendritic cell precursors will be most useful for enhanced cross-priming and enhanced cross-tolerance; phagocytes other than dendritic cells may be more useful for enhancing clearance of apoptotic cells. However, the invention is not so limiting as to categorize particular cell types for particular uses, because by modifying the expression of and selection of the particular apoptotic cell receptors, and providing the proper milieu, cells of one type may be manipulated in accordance with the teaching herein to serve an altered function.

The present invention generally embraces any and all receptors the increased expression of which enhances the capture of apoptotic cells. These receptors include both naturally-occurring receptor proteins and complexes, as well as chimeric and mutant receptors, referred to herein as modified receptors. Examples of known receptors include, but are not limited to, members of the Fc receptor family, members of the scavenger receptor family, CD14, members of the ABC-1 family of transporters, members of the C-type lectin family, an integrin receptor β subunit other than β₁, and an integrin receptor heterodimer other than that comprising β₁. Non-limiting examples of members of these families are listed here for purposes of illustration; the skilled artisan will be amply aware of the extent of such family members from the literature. Thus, members of the Fc receptor family include FcγRI, FcγRIIA, FcγRIIB or FcγRIII α-chain or the signaling sequence of the Fc γ-chain; members of the scavenger receptor family include SR-A, CD36, C1qR; CD14; members of the ABC-1 family of transporters ; and members of the C-type lectin family include the macrophage mannose receptor, DEC-205, DECTIN-1, DECTIN-2. Integrin receptor β subunits other than β₁ include β₂, β₃, and β₅; integrin receptor heterodimers other than that comprising β₁ include αᵥβ₂, αᵥβ₃, and αᵥβ₅. Moreover, the present invention includes chimeric receptors, in particular, a β integrin subunit in which the extracellular portion comprises that from β₅, and a signaling portion derived from a β subunit other than from β₁, or from a member of the Fc receptor family (see above); or a member of the C-type lectin family (see above). The signaling (tail) portion of the Fc receptor family may be the FcγRI α-chain, FcγRIIA α-chain, FcγRIIB α-chain, or FcγRIII α-chain, or any Fc γ-chain with properties similar to that of β₅. Other extracellular domains with β₅-like properties are likewise included.

A preferred apoptotic cell receptor is the integrin receptor, and more particularly, αᵥβ₅. As will be seen in the examples below, increased expression of the αᵥβ₅ receptor enhances apoptotic cell capture, cross-presentation of antigen, and cross-priming of T cells to the antigen (see below). As also will be seen below, in one embodiment of the invention, enhanced expression of the αᵥβ₅ receptor may be provided by genetically modifying a cell to increase expression of the β₅ subunit only; whether native, chimeric or mutant; as increased expression of this subunit alone will recruit the αᵥ subunit to provide integrin heterodimers on the cell surface.

In another embodiment, a chimera may be prepared with the αᵥ portion of an integrin receptor heterodimer comprising a chimeric β subunit other than β₁, or an integrin receptor heterodimer comprising a mutant β subunit, as described above.

In this aspect of the invention, the genetically modifying the phagocyte may be carried out by any of the aforementioned methods, including infection, transfection or gene transfer. The use of a viral vector is preferred.

In the practice of this aspect of the invention, a phagocytic cell with enhanced expression of an apoptotic-cell receptor is provided, and the phagocytic cell is exposed to an apoptotic cell comprising an antigen. The invention is also drawn to genetically-modified phagocytes with enhanced ability to capture an apoptotic-cell-delivered antigen, as prepared by the foregoing methods.

This aspect of the invention may be carried out in vitro, or ex vivo, using cells derived from a patient. In-vivo gene therapy to induce receptor expression in cells or tissues within the body is also embraced herein. As the enhanced capture of apoptotic cells by the foregoing method is the starting point for all of the other methods and uses of the invention, this method embraces all of the embodiments described below for particular uses of the enhanced capture. Furthermore, as noted above, more than one antigen may be delivered to phagocytes by these methods, such as by providing apoptotic cells containing more than one antigen, or by exposing the phagocytic cells to two or more apoptotic cell populations, each of which contains a different antigen.

### Enhanced Cross-Presentation of Dendritic Cells by Apoptotic Cell-Delivered Antigens

One particularly useful aspect of enhanced apoptotic cell capture by any of the foregoing methods is the enhanced cross-presentation of the antigen or antigens delivered to the phagocyte by the captured apoptotic cell(s). By selecting the appropriate combination of the type of phagocytic cell and the apoptotic cell receptor, the captured antigen can be trafficked and cross-presented to T cells to achieve such effects, described in more detail below, as enhanced cross-priming, or enhanced cross-tolerance to the antigen. In the case of dendritic cells, appropriate apoptotic cell receptors capable of enhancing cross-presentation include a member of the Fc receptor family, a member of the scavenger receptor family, a member of the ABC-1 family of transporters, a member of the C-type lectin family, an integrin receptor β subunit other than β₁ or β₃, an integrin receptor heterodimer comprising a β subunit other than β₁ or β₃, an integrin receptor heterodimer comprising a chimeric β subunit other than β₁ or β₃, and an integrin receptor heterodimer comprising a mutant β subunit. The chimeric β subunit with enhanced cross-presentation may comprise the extracellular domain of integrin β₅ and a signaling domain of integrin β₅ or of the Fc receptor FcγRI, FcγRIIA, FcγRIIB or FcγRIII α-chain. Although dendritic cells are preferred, other phagocytic cells expressing the foregoing receptor and demonstrating enhanced cross-presentation are embraced herein.

The various selections of receptors within the aforementioned group are those as described in more detail hereinabove. Methods for enhancing the expression of such receptors is also as described above.

As will be noted throughout this application, enhanced cross-presentation has numerous utilities in the prophylaxis or treatment of a variety of conditions and diseases related to the immune system. For example, enhanced cross-presentation of antigen resulting in enhanced cross-priming of T cells results in increased recognition and killing of cells expressing the antigen, which if a tumor or viral antigen, results in enhanced killing of tumor cells or virally-infected cells. Cross-presentation of antigen for the purpose of suppression of T cell activity is therapeutically beneficial in turning off the immune response to, for example, an auto-antigen responsible for an autoimmune disease. A further example of enhanced suppression is in the prophylaxis or treatment of transplanted organ rejection, wherein the expected immune response against foreign antigens is suppressed in advance of the transplant, or rejection is diminished.

As noted above, enhanced cross-presentation may be carried out in the context of ex-vivo treatment of phagocytic cells isolated from an individual, for later reintroduction, or using exogenous phagocytes. Such methods are embraced by the further examples below.

### Enhanced Clearance

Enhanced apoptotic cell capture in the absence of antigen cross-presentation results in clearance of the apoptotic cell and the associated antigen. In particular, phagocytic cells other than dendritic cells may be employed, by the genetic modification described herein, to increase or enhance the uptake of apoptotic cells, but not cross-present the antigen because of the characteristics of the particular apoptotic cell receptor on the modified phagocytes. In particular, the various receptors described hereinabove, in combination with phagocytes which are not dendritic cells, results in such enhanced clearance. Thus, the apoptotic cells and antigen contained therein are degraded without eliciting an immune response to the antigen. Therefore, such cells include professional phagocytes, for example an antigen presenting cell, and by way of further example a macrophage, neutrophil, or B cell; or a nonprofessional phagocyte, such as but not limited to a keratinocyte, a fibroblast, an epithelial cell or an endothelial cell. The phagocyte may be a human or non-human phagocyte. Non-human cells including various insect or other non-mammalian cells lines with the foregoing properties may also be modified in accordance with the teachings herein, for in vivo use to degrade apoptotic cells.

Appropriate receptors for achieving this aspect of the invention include a member of the Fc receptor family, a member of the scavenger receptor family, CD14, a member of the ABC-1 family of transporters, a member of the C-type lectin family, an integrin receptor β subunit other than β₁, an integrin receptor heterodimer comprising a β subunit other than β₁, an integrin receptor heterodimer comprising a chimeric β subunit other than β₁, and an integrin receptor heterodimer comprising a mutant β subunit. Examples of the particular receptors within this group are described above, as well as means for achieving the genetic modification.

The utility of phagocytic cells with enhanced clearance of apoptotic cells is found particularly in diseases or conditions in which defective clearance of apoptotic cells results in an unwanted immune response to the cells, such as occurs in episodic flares of SLE. By providing the individual or the affected tissue with phagocytes in accordance with this aspect of the invention, enhanced clearance of apoptotic cell corpses may be achieved.

The method of this aspect of the invention may be performed by first genetically modifying an aforementioned phagocyte to increase expression of an apoptotic-cell receptor capable of enhancing capture of apoptotic cells and promoting degradation of said antigen; and then introducing the genetically-modified phagocyte into diseases tissue of an individual. In another embodiment, gene therapy to transfect phagocytes in vivo, and in particular within affected tissues in which enhanced clearance is desired, may be achieved.

### Enhanced Cross-Priming of Dendritic Cells by Apoptotic Cell-Delivered Antigens

As mentioned above, one utility of enhanced delivery of an apoptotic cell antigen is the enhanced cross-priming of T cells by dendritic cells to enhance the formation of antigen-specific CTLs. In the practice of this aspect of the invention, the first step is genetically modifying dendritic cells or precursors thereof to increase expression of an apoptotic-cell receptor capable of promoting capture of apoptotic cells and trafficking internalized apoptotic material thereby enhancing cross-priming of T cells, or alternately, genetically modifying dendritic cells to express a modified apoptotic cell receptor with enhanced capture ability; and then exposing the genetically-modified dendritic cells to an apoptotic cell comprising at least one antigen, in the presence of immunostimulatory exogenous factor(s) or antigen-specific CD4 helper T cells, wherein the dendritic cells result in having enhanced ability promote the formation of antigen-specific CD8 cells. In a subsequent step, contact of the dendritic cells with T cells promotes or enhanced antigen-specific T cell formation. As noted above, a particular subset of the apoptotic-cell receptors capable of increasing apoptotic cell capture is applicable to this aspect of the invention, namely, an immunostimulatory member of the Fc receptor family (i.e., having an ITAM motif), a member of the scavenger receptor family, a member of the C-type lectin family, a β₅ integrin receptor subunit, an integrin receptor heterodimer comprising β₅, such as αᵥβ₅, an integrin receptor heterodimer comprising a chimeric β subunit with a β₅ signaling domain or a Fc signaling domain capable of cross-priming, and an integrin receptor heterodimer comprising a mutant β subunit. Fc signaling domains capable of cross-priming include the FcgRI α-chain, FcgIIA α-chain or FcgRIII α-chain.

The dendritic cells useful for this aspect of the invention include myeloid dendritic cells or lymphoid myeloid dendritic cells.

The exogenous immunostimulatory factors needed to promote the enhancement of cross-priming include but are not limited to at least one of CD40 ligand, TRANCE, TRAIL, OX40, or an alternate member of the TNF superfamily, thalidomide, or another agent that participates in cross-priming CTLs.

As noted above, the utility of this aspect of the invention is in the enhancement of the killing of tumor cells, virally-infected cells, and destruction of other cells bearing antigens, such as but not limited to bacterial infections, protozoan, such as malaria, listeriosis, microbial infections, viral infections such as HIV or influenza, and cancers or malignancies. A preferred embodiment is the enhancement of CTLs towards cancer cells. Non-limiting examples of cancers and malignancies include melanoma, cancer of the prostate and cervix, and small cell lung cancer, to name only a few.

Moreover, this aspect of the instant invention also may result in the enhanced formation of antigen-specific CD4 helper cells.

In a preferred embodiment of this aspect of the invention, at least the following steps may be carried to produce enhanced CTLs to at least one preselected antigen:

a) obtaining a source of dendritic cells or precursors thereof;

b) genetically modifying the dendritic cells or precursors thereof with at least one apoptotic-cell receptor capable of promoting capture of apoptotic cells and enhancing cross-priming of said at least one antigen, as described above;

c) exposing the genetically-modified dendritic cells or precursors thereof to apoptotic cells expressing the at least one antigen in the presence of at least one of the following compositions:

i) an agent capable of both facilitating cross-priming and maturing said dendritic cell; or

ii) the combination of at least one agent capable of facilitating cross-priming but not capable of maturing said dendritic cell, and at least one agent capable of inducing dendritic cell maturation but not capable of facilitating cross-priming;

d) optionally isolating said dendritic cells; and

e) administering said dendritic cells to a patient in need thereof.

In a more specific aspect of the invention, a method for enhancing cross-priming of T cells by dendritic cells or precursors thereof using an apoptotic-cell-delivered antigen is carried out by following at least the following steps:

(a) genetically modifying dendritic cells or precursors thereof to express or increase expression of an integrin heterodimer which may be one of the following:

i) αᵥβ₅;

ii) a heterodimer of αᵥ and a chimeric β subunit comprising an extracellular β₅ domain and an Fc γ signaling domain with an ICAM repeat, such as FcγRI, FcγRIIA, or FcγRIII α-chain signaling domain;

iii) a heterodimer of αᵥ and a chimeric β subunit comprising an extracellular β₅ domain and an integrin β₃ or β₅ signaling domain;

iv) a chimeric β subunit alone comprising an extracellular β₅ domain and an integrin β₃ or β₅ signaling domain; or

v) a chimeric β subunit alone comprising an extracellular β₅ domain and an a Fc FcγRI, FcγRIIA, or FcγRIII α-chain signaling domain;

(b) exposing said genetically-modified dendritic cells to an apoptotic cell comprising an antigen in the presence of at least one immunostimulatory exogenous factor or antigen-specific CD4 helper T cells,

wherein said dendritic cells or precursors thereof have enhanced ability to form antigen-specific CD8 cells. As noted above, the immunostimulatory exogenous factor may be at least one of CD40 ligand, TRANCE, TRAIL, OX40, or an alternate member of the TNF superfamily, thalidomide, or another agent that may participate in cross-priming of CTLs. Subsequent optional isolation of the dendritic cells and introduction into the body enhances antigen-specific T cell formation.

The dendritic cells may be myeloid dendritic cells or lymphoid dendritic cells. Preferably the dendritic cells are human dendritic cells, but other cells including non-human cells with the properties of dendritic cells may be used (xenogeneic antigen presenting cells), such as various insect (e.g., Drosophila) or mammalian (e.g., murine) or other non-mammalian cells lines with the foregoing properties may also be modified in accordance with the teachings herein, for in vivo use to capture apoptotic cells (i.e., non-human antigen presenting cells). An example is murine antigen presenting cells which express MHC I, in combination with costimulatory molecules such as B71 or other adhesion molecules critical for such cells to interact with T cells.

The dendritic cells may be obtained from any appropriate source and by any appropriate method, such as but not limited to allogeneic cord blood, bone marrow biopsy, bone marrow-derived dendritic cell precursors, isolated dendritic cell precursors, cells obtained by leukapheresis, and dendritic cells mobilized from the bone marrow to the peripheral blood. Such methods for isolating dendritic cells are found in the aforementioned literature.

The methods may preferably be carried out in vitro or ex vivo, and after exposure of the dendritic cells to the apoptotic cells under the appropriate conditions, the dendritic cells may be introduced or reintroduced into the patient where interaction with T cells results in an enhanced response. If necessary, the dendritic cells may be isolated after ex-vivo treatment by standard methods for isolating dendritic cells, such as methods known in the art.

The agent capable of both facilitating cross-priming and maturing said phagocytic cell may be CD40 ligand, a member of the TNF superfamily, and IL-1β. The agent capable of facilitating cross-priming but not capable of maturing said phagocyte may be TRANCE, thalidomide or IL-12. The agent capable of inducing phagocyte maturation but not capable of facilitating cross-priming is monocyte conditioned medium, IL-6, TNF-α, IL-1β or PGE₂.

Preferably, the integrin receptor β subunit is β₅; or the integrin receptor heterodimer is αᵥβ₅. As noted above, it has been found by the inventors herein that genetically modifying dendritic cells to increase expression of the integrin receptor β subunit alone results in recruitment of the a subunit, thus, the foregoing methods in which an integrin receptor heterodimer is provided in a cell may be achieved by, for example, transfecting the β subunit gene only, whether native or a chimeric protein. Thus, the integrin receptor heterodimer or β subunit comprises a chimeric β subunit with an extracellular β₅ domain and an signaling domain selected from integrin receptor β₅, FcγRI α-chain, FcγIIA α-chain or FcγRIII α-chain.

The aforementioned utilities of an enhanced CTL response is applicable to this aspect of the invention.

### Enhanced Cross-Tolerance of Dendritic Cells

The enhanced cross-presentation of an apoptotic cell-delivered antigen as described above may also be used to enhance the suppression of a CTL response, in particular using dendritic cells, when the appropriate combination of apoptotic cell receptor and microenvironment is employed. The method generally is carried out by providing genetically modifying dendritic cells or precursors thereof with increased expression of an apoptotic-cell receptor capable of promoting capture of apoptotic cells and enhancing cross-tolerance of T cells; and exposing the genetically-modified dendritic cells or precursors to an apoptotic cell comprising an antigen in the presence of immunosuppressive exogenous factors or in the absence of the combination of antigen-specific CD4 helper T cells and immunostimulatory exogenous factor(s). This method generates dendritic cells having increased ability to tolerize antigen-specific CD8 cells.

The apoptotic-cell receptor capable of enhancing cross-tolerance of T cells may be an integrin receptor heterodimer with a β₂ subunit, a cross-tolerizing member of the FcR family, i.e., one that contains an ITIM motif, or a chimeric integrin receptor β subunit with an extracellular β₅ domain and a signaling domain that is either from integrin receptor subunit β₂ or FcγRIIB α-chain. The immunosuppressive exogenous factor may be , for example, TGF-β, IL-10, IL-4, IL-5, IL-13, FK506 (tacrolimus) or an agent that binds to FKBP12.

By carrying out the method, the cross-tolerance results in a decrease in autoreactive T cells to the antigen. Thus, an autoimmune disease may be treated by enhancing the tolerization of T cells specific for autoantigens, by carrying out the aforementioned method. Such diseases as psoriasis, Crohn's disease, rheumatoid arthritis, and multiple sclerosis are exemplary of diseases that may be treated, but other autoimmune diseases or diseases with an autoimmune component are embraced herein.

Another particularly utility of the method is in the prophylaxis of the immune response prior to organ transplant to obviate an immune response to a transplanted antigen. The recipient prior to the transplant may be tolerized to the donor antigens, by carrying out the instant methods using the donors antigens delivered by apoptotic cells, or apoptotic donor cells themselves.

The method also results in tolerizing of CD4 helper cells to the antigen.

In a more specific embodiment of the cross-tolerizing aspect of the invention, the method for enhancing cross-tolerance to an apoptotic-cell-delivered antigen by dendritic cells or precursors thereof may be carried out by following at least the following steps:

(a) genetically modifying dendritic cells or precursors thereof to express an apoptotic cell receptor capable of enhanced capture, or increase expression, of an integrin heterodimer comprising

i) a heterodimer of αᵥ and a chimeric β subunit comprising an extracellular β₅ domain and a signaling β₂ domain or a ITIM-motif-containing γ-chain signaling domain, such as the FcγRIIB domain;

ii) a chimeric β subunit alone comprising an extracellular β₅ domain and a signaling β₂ domain; or

iii) a chimeric β subunit alone comprising an extracellular β₅ domain and a signaling FcγRIIB domain or other γ-chain with an ITIM motif; and

(b) exposing the genetically-modified phagocyte to an apoptotic cell comprising an antigen in the presence of immunosuppressive exogenous factor(s) or in the absence of the combination of antigen-specific CD4 helper T cells and immunostimulatory exogenous factor(s).

As a result, the dendritic cells have a reduced ability to cross-prime T cells with the antigen. The immunosuppressive exogenous factor may be TGF-β, IL-10, IL-4, IL-5, IL-13, FK506 (tacrolimus) or an agent that binds to FKBP12, by way of non-limiting example. Alternately, the method may be carried out in the absence of both antigen-specific CD4 helper T cells and immunostimulatory exogenous factors, such as those described hereinabove.

In a still more specific embodiment of the invention, a method for suppressing the immune response in a mammalian patent to a preselected antigen is carried out by:

a) obtaining a source of dendritic cells of precursors thereof;

b) genetically modifying the dendritic cells with an apoptotic-cell receptor capable of promoting apoptotic cell capture, cross-presentation of an apoptotic cell-delivered antigen and promoting cross-tolerance of the antigen;

c) exposing the genetically-modified dendritic cells to apoptotic cells expressing the antigen in presence of immunosuppressive exogenous factor(s) such as TGF-β, IL-10, IL-4, IL-5, IL-13, FK506 (tacrolimus) or an agent that binds to FKBP12 or in the absence of the combination of CD4 helper T cells and immunostimulatory exogenous factor(s);

d) optionally isolating the dendritic cells; and

e) administering said dendritic cells to a patient in need thereof.

The immunostimulatory factors are those as described hereinabove with respect to the cross-priming of dendritic cells. Appropriate apoptotic-cell receptors capable of enhancing cross-tolerance of T cells is an integrin receptor heterodimer with a β₂ subunit or a chimeric β subunit with an extracellular β₅ domain and an signaling domain that is integrin receptor β₂ subunit or the FcγIIB α-chain.

The dendritic cells prepared by the foregoing method may be isolated before introduction to the patient, for example, by methods as described in the pervious section.

### Recruitment of Integrin Receptor Heterodimers by Genetic Modification with β Subunit Only

As mentioned above, the present inventors found unexpectedly that increasing the expression of an integrin receptor heterodimer in a cell may be achieved by genetically modifying the cell with only the integrin receptor β subunit, whether native, chimeric or mutant. Mutant as defined herein refers to any mutation which results in the ability of the molecule to signal like the native receptor. Thus, for the various methods of the present invention, as well as other methods in which increased expression of integrin heterodimers is desirable, a gene, vector, or other construct which results in the expression only the β subunit needs to be introduced into the cell, as it is capable of achieving the upregulation of the a subunit and the appearance of heterodimers on the cell surface.

Therefore, constructs comprising the various native β subunits described herein, as well as the chimeric or mutant β subunits, may be transfected or otherwise introduced into cells for the purpose of increasing the expression of a heterodimer comprising the introduced β subunit and an alpha subunit already present in the cell.

### New Model for Evaluating Apoptotic Cell Delivery to Phagocytes

As mentioned above, it was found by the inventors herein that among a variety of cells tested, 293T cells have characteristics including a receptor profile similar to dendritic cells and thus are capable of phagocytosing apoptotic cells. Thus, such cells are useful for in-vitro studies of enhanced phagocytic cell capture of apoptotic cells.

The basis for the foregoing methods of the invention will be apparent from the following Examples which illustrate the various aspects described above. These studies describe a novel role for the β₅ integrin as a receptor for the phagocytosis of apoptotic cells and defined the first molecular feature distinguishing DCs in their handling of apoptotic material. Moreover, receptor utilization for the capture of apoptotic cells has been shown to influence the immunologic outcome. As apparent from the incomplete inhibition of phagocytosis observed when using anti-αᵥβ₅ monoclonal antibodies, other receptors are involved in the uptake of apoptotic cells (8). The list of receptors include: FcR, complement receptors 3 and 4 (CR3, CR4—also known as αₘβ₂ (MAC-1) and αₓβ₂, respectively), ABC1, and scavenger receptor A family members (SR-A) (34-36). Additionally, putative receptors include the macrophage mannose receptor (MR) and a still undefined phosphatidylserine receptor (37, 38). Some of the effects of DC's capturing antigen via these various receptors have been defined. For example, apoptotic cells opsonized with anti-phospholipid antibodies enter via the FcR, resulting in DC maturation and the priming of antigen-specific T cells (34). Conversely, utilization of the CR3 in the absence of FcR activation likely results in an immunosuppressive event (39, 40). The present invention extends to these various other receptors which the skilled artisan will recognize as being useful for the same purposes.

The various aspects of the invention have particular utilities in the prophylaxis and treatment of various conditions and diseases, as alluded to or described above. The enhancement of immunological activation or suppression of a cytotoxic T cell response, and corresponding effects on helper T cells, bears utility in all manipulations of the immune system where CTLs are involved. The methods of the invention can be used in an ex vivo procedure in which dendritic cells are isolated from the body, treated as described herein to genetically modify apoptotic cell receptors, exposed to apoptotic cells in a microenvironment conducive to the desired outcome (e.g., priming, tolerizing), and then reintroduced to the body where the desired effect on CTLs is achieved.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE 1

### The Human Kidney Epithelial 293T Cell Line Provides an Appropriate Model Cell to Study Dendritic Cell-Mediated Phagocytosis

A model system was developed for screening candidate signaling proteins that may be involved in β₅ mediated phagocytosis. Various cells lines including HeLa cells, 3T3 cells, COS cells and 293T cells were all tested for their ability to capture apoptotic cells in a manner akin to dendritic cells. It was determined that 293T cells, a human kidney epithelial cell line, are an appropriate model system. The ability of 293T cells to engulf apoptotic material was first determined using a FACScan®-based phagocytosis assay (method described in [8]). Human T cells, freshly isolated from peripheral blood mononuclear cells (PBMCs) were employed as a source of apoptotic cells due to the fact that they are non-adherent and can be easily distinguished from 293T cells by FACS® based on size. PMBCs used in all experiments are acquired from leukocyte paks, which are commercially available through the Red Cross Blood Center (NYC, N.Y.).

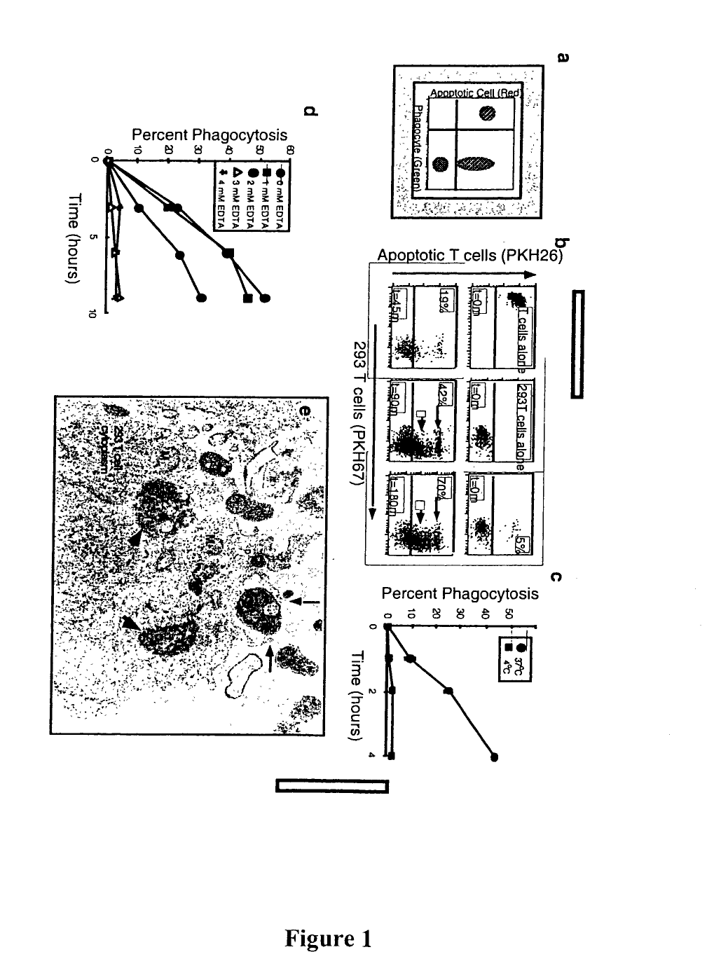

The T cells were labeled with a red fluorescent cell linker (PKH-26GL, Sigma) and irradiated with 120 mJ/cm2 UV-B to trigger apoptosis. Apoptotic death was tracked using various techniques. By 5-6 hours, the majority of T cells were annexin V⁺/propidium iodide⁻, indicating that phosphatidylserine is exposed on the outer leaflet of an intact plasma membrane. By 10-12 hours, cells were TUNEL positive and at 24-36 hours, they began to undergo secondary necrosis as characterized by Trypan Blue inclusion. The 8-10 hour time point was employed to ensure that further experiments utilized apoptotic cells with intact plasma membranes. To establish co-cultures with the phagocyte, the 293T cells were labeled with a green fluorescent cell linker (PKH-67GL) and added to the wells containing the dying T cells. After various time intervals, co-cultures were analyzed by FACS®, and double positive cells indicated that the 293T cell had engulfed an apoptotic cell (FIG. 1a). As noted in FIG. 1, 293T cells efficiently phagocytose apoptotic cells. Shown is a representative time course of 293T cells engulfing apoptotic T cells. Values in the top left corner indicate the percent of 293T cells which are double positive. Note, prior to running the FACS® analysis, co-cultures are placed into a 5 mM EDTA solution to eliminate cells bound but not internalized by the phagocyte (b). To demonstrate that the apoptotic T cells were being phagocytosed, co-cultures were incubated at low temperature (c), with Cytochalasin D, or with various concentrations of EDTA (d). Phagocytosis was also evaluated by electron microscopy (e). Shown is a representative image with two apoptotic cells/bodies within the 293T cell (large arrowheads) and multiple apoptotic cells/bodies attached at the plasma membrane. One in particular appears to have triggered membrane ruffling (small arrows). Key: AC, apoptotic cell/body. M, mitochondria. Similar studies were also performed with EL4 (murine) cells and human macrophages.

Just prior to running each sample, the co-cultured cells were placed in a 5 mM EDTA solution and vortexed to ensure that internalization was being measured and not merely binding of the T cell to the 293T cell plasma membrane. As expected, due to the 293T cells being non-professional phagocytes (41), it was found that they are less efficient than DCs at capturing the apoptotic T cells. To compensate for this feature, the number of apoptotic cells was increased, and it was found that at ratios of 10:1 apoptotic cells : 293T cells, 40-70% uptake was consistently achieved within 2-4 hours (FIG. 1b). The high dose of apoptotic cells does not affect the survival of the 293T cells, and at this ratio the kinetics of uptake and percent of 293T cells that capture an apoptotic cell matches that found in apoptotic cell : DC co-cultures.

To establish that the FACS® assay was measuring phagocytosis, the assay was carried out at 4° C. and in the presence of inhibitors of phagocytosis. Both low temperature (FIG. 1c), and cytochalasin D, an inhibitor of cytoskeletal function, blocked uptake. Phagocytosis by the 293T cells also requires divalent cations as when EDTA was added during the 293T cell-apoptotic cell co-culture period, phagocytosis was inhibited (FIG. 1d). To visually confirm the uptake recorded by FACS®, cytospins were prepared of the dyed co-cultures. The frequency of uptake correlated with that measured on FACS®. Electron microscopy was performed on co-cultures of 293T cells and apoptotic T cells (FIG. 1e). In the representative image shown, an apoptotic cell/body (AC) is seen just prior to being engulfed by a 293T cell, with characteristic ruffling of the plasma membrane evident (FIG. 1e, arrows). Following phagocytosis, apoptotic cells/bodies were found in phagolysosomes of the 293T cells (FIG. 1e, arrowheads).

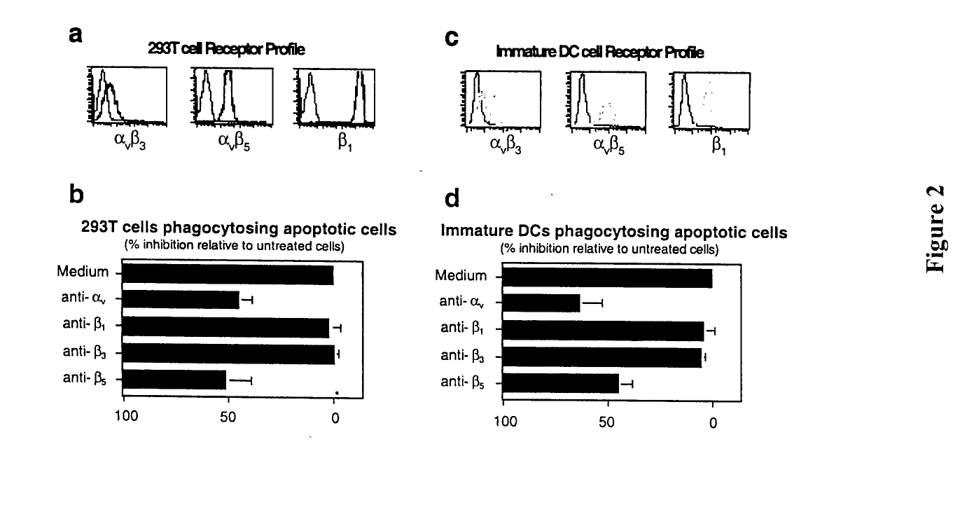

To experimentally address whether 293T cells employ a mechanism similar to DCs for recognition and internalization of apoptotic cells, the surface receptor profiles of 293T and DCs were compared using commercially-available monoclonal antibodies, mAbs (FIGS. 2a,c). 293T cells and immature DCs were labeled with various monoclonal antibodies to determine the surface receptor profile. The black lines indicate staining with an isotype matched control antibody. Both cell types express high levels of β₅ and low levels of β₃ as detected with heterodimer-specific antibodies (a, b). To test for a functional role for β₅ in these two cells lines, apoptotic T cells were labeled with PKH26-GL, followed by irradiation using a 60UVB lamp, calibrated to provide 240 mJ cm-2 in 2 minutes, and sufficient for the induction of apoptosis. After 6-8 hours 293T or cells immature DCs were dyed with PKH67-GL and pre-treated with 50 microg /ml of various monoclonal antibodies for 30 minutes, then added to the wells containing the apoptotic cells at ratios of 1:10 and 1:1, respectively. Sixty to 90 minutes later, cells were analyzed by FACS® for double positive cells. Phagocytic uptake is reported as a percentage of untreated cells. Maximal phagocytosis ranged from 44-52% in both cell types. Results from 2-3 experiments were averaged and means plotted +SD (c, d).

Both 293T cells and DCs have low receptor density of the β₃ integrin heterodimer and high expression of the β₅ integrin receptor. Additionally, β₅-specific mAb (clone B5-IVF2), but not the anti-β3 antibody (clones SZ21 & RUU-PL7F12 were both tested), inhibits the 293T cell's ability to phagocytose apoptotic cells (FIG. 2b). Notably, the 50-60% inhibition that was observed matches that achieved in DCs (FIG. 2d) (8).

One relevant difference between the 293T cells and DCs in their phagocytic receptor profile is the expression of CD36 (FIGS. 2a,b). While >90% of the immature DCs demonstrate high levels of CD36, only ˜15-20% of the 293T cells express this receptor on the surface. This difference is believed to account for the finding that the 293T cells are less efficient than the DCs in capturing apoptotic cells, as CD36 is known to enhance interactions between v integrins and their ligand (42, 43). Furthermore, there is significant evidence that CD36 acts as a co-receptor for the β₅ integrin in other biologic systems (e.g. angiogenesis) (18, 43-45).

### EXAMPLE 2

### Signaling via the β₅ Integrin Receptor is Critical for the Internalization of Apoptotic Cells

To establish a direct role for the β₅ integrin receptor in the phagocytosis of apoptotic cells, a dominant-negative form of the receptor needed to be defined which could be introduced into the 293T cells. The cytoplasmic tail of the β₅ subunit possess two NXXY internalization motifs (46).

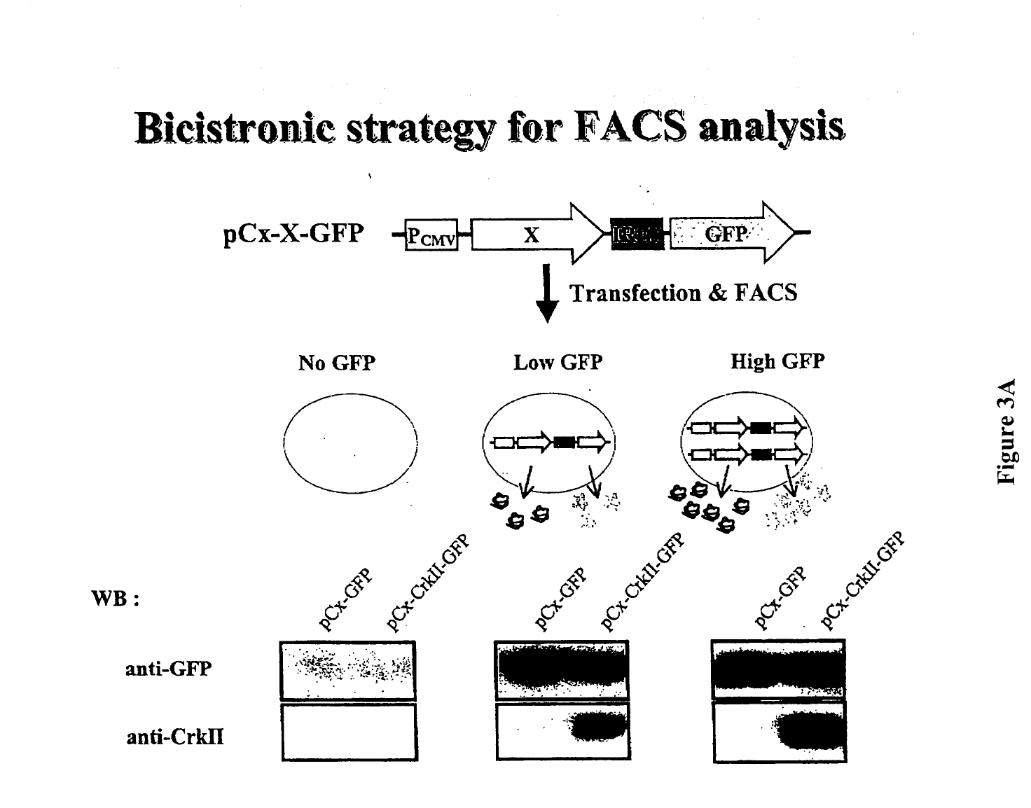

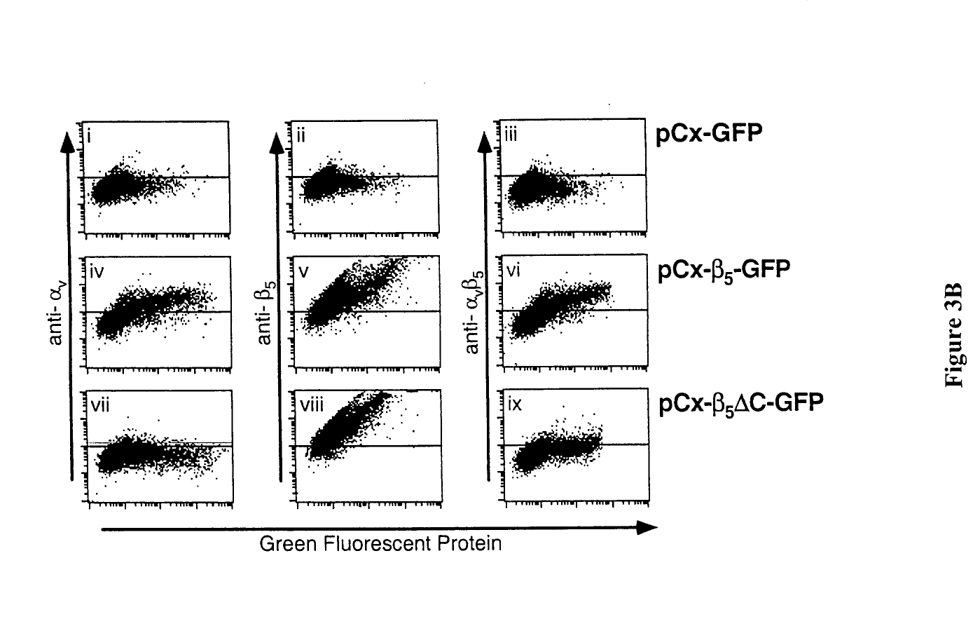

Therefore putative dominant-negative mutant was constructed by deleting the cytoplasmic tail (β₅ΔC). Bicistronic vectors were designed so that a single mRNA transcript contained either the β₅ΔC or wild type β₅, as well as the gene for green fluorescent protein (GFP), expressed by an internal ribosomal entry site, IRES (FIG. 3a).

A schematic representation of construct used for developing bicistronic vectors is shown in the figure. The CMV promoter is used to drive the expression of a single mRNA containing the gene of interest and an IRES-GFP (a). Using this strategy, wild type β₅ or β₅ΔC can be expressed and GFP expression can be used as a measure of receptor density. Shown is a correlation between GFP intensity and the surface expression of β₅, and the β₅ heterodimer when using empty vector (pCx-GFP), β₅-GFP or β₅ΔC-GFP. This strategy permits the measurement of phagocytosis and cross-presentation as a function of gene expression in a single-cell based FACS® assay (b).

In this way, equivalent amounts of the desired gene and GFP are expressed, thus making it possible to design a single-cell assay in which β₅ΔC or wild type β₅ expression levels can be accurately measured based on the relative intensity of green fluorescence, as determined by FACS® (FIG. 3b). Surprisingly, when evaluating the correlation of surface expression of β₅ and GFP (FIG. 3b, panels ii, v, viii), it was discovered that overexpression of wild-type β₅ resulted in increased surface expression of β and thus higher levels of surface β₅ (FIG. 3b, panel iv, vi). This phenomenon was dependent on the cytoplasmic tail of the β₅ receptor, as this effect was not observed when β₅ΔC was employed (FIG. 3b, panel vii, ix). This observation was fortuitous as it allowed measurement not only the effect of expressing a β₅ dominant-negative, but also permitted determining the outcome of increased β₅ expression on the ability to phagocytose an apoptotic cell. Such observations will be pivotal in the designing of adenoviral constructs for the delivery of functional β₅ complexes to DCs.

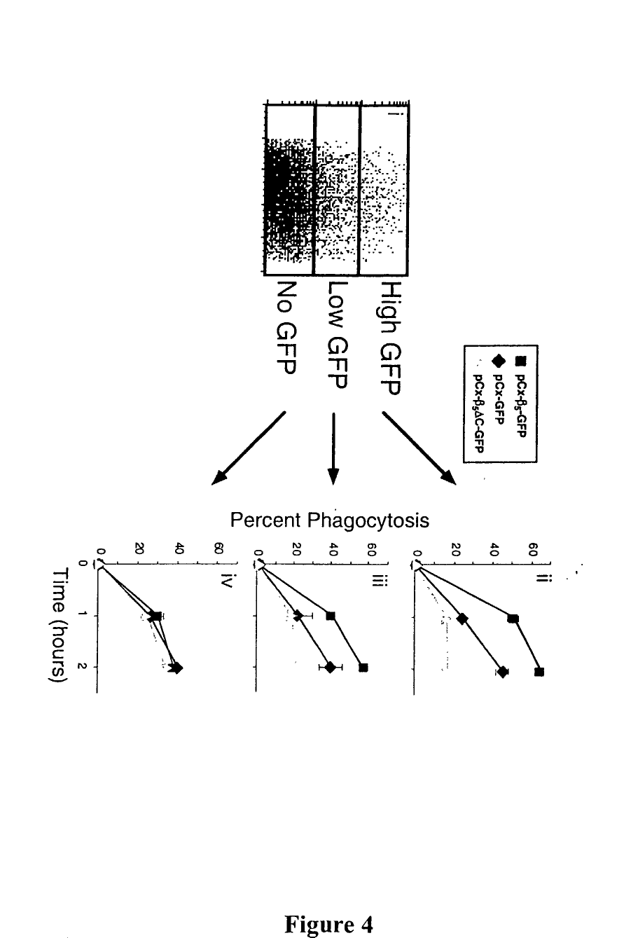

293T cells were transfected with the β₅ΔC-GFP, β₅-GFP or a control vector expressing only GFP, using the retroviral bicistronic construct pCX, which utilizes a CMV promoter to drive high level of expression. Cells were transfected using the lipofectamine reagentTM, and allowed to recover for 4-5 days after which they were incubated with apoptotic PKH-26GL labeled T cells. At various time intervals, co-cultures were analyzed by FACScan® analysis, allowing us to evaluate the effect of β₅ΔC and wild type β₅ overexpression on the uptake of apoptotic cells. The 293T cells were gated based on the expression level of GFP (FIG. 4a)

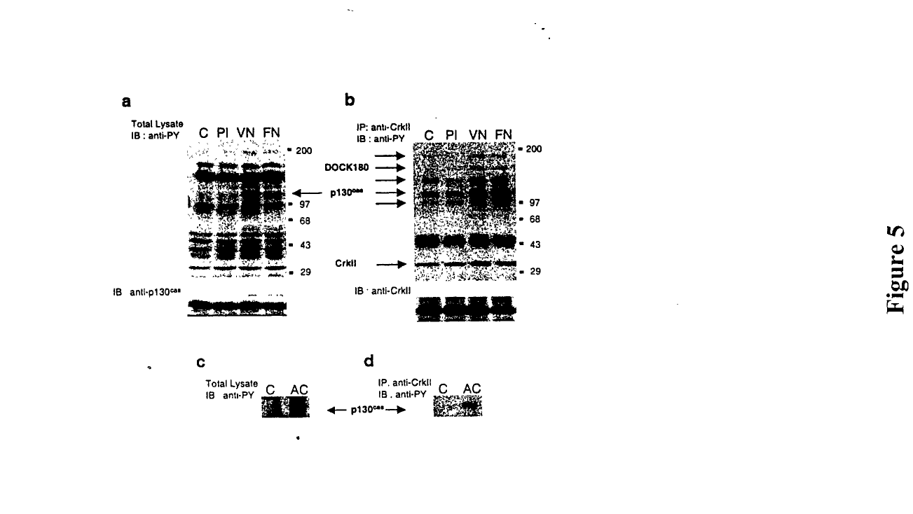

FIG. 5 shows that β₅ integrin expression regulates phagocytosis of apoptotic cells. Bicistronic vectors encoding wild type β₅ and β₅ΔC were transfected into 293T cells. These cells were allowed to recover for 4-5 days after which they were placed in co-culture with red labeled apoptotic T cells at a ratio of 1:10. At one hour intervals, uptake was measured using the FACS® based phagocytosis assay. By creating regions containing single cells expressing various levels of GFP (a) it was possible to measure the effects of graded doses of β₅ and β₅ΔC expression on the 293T cells ability to phagocytose apoptotic cells (b-d).

When no GFP was expressed, no overexpression of β₅ or wild type β₅ occurred, and no difference in the cells ability to phagocytose an apoptotic cell was observed (FIG. 4d). As more β₅ΔC was overexpressed, the 293T cells ability to capture the apoptotic cell was diminished in a dose dependent manner (FIGS. 4b,c). Conversely, when wild type β₅ overexpressed, the 293T cells were more efficient at engulfing the dying T cells, and again this effect was titratable with respect to the expression level of the β₅ integrin (FIGS. 4b,c). Prior experiments have all relied on monoclonal antibodies to demonstrate that inhibition of integrin receptor/apoptotic cell interactions results in decreased phagocytosis, and therefore could not exclude the possibility that the integrin is merely acting to bind the dying cell (8, 47). This is the first demonstration that integrins play an active role in the internalization of apoptotic cells, presumably by initiating a signaling event, resulting in cytoskeletal rearrangement.

### EXAMPLE 3

### The β₅ Integrin Receptor Subunit Recruits the Crk/DOCK180 Molecular Complex to the Plasma Membrane

These studies investigated the downstream events initiated by β₅ activation. Based on data relevant to other integrin receptors, it was postulated that a tyrosine kinase signaling pathway is involved (reviewed in 48). As will be shown, tyrosine phosphorylation of p130cas was evident 30 minutes after plating, when most of the 293T cells began to attach to the VN. 293T cells were plated on plastic coated plates (Pl) or plates coated with fibronectin (FN) or vitronectin (VN). VN is an extracellular matrix protein which is known to bind the β₅ heterodimer (49). After 30 min., cells were lysed in HNTG buffer (contains 1% Triton X). Cell nuclei were removed by centrifugation and total lysate was run on SDS-PAGE. Anti-phosphotyrosine antibody (anti-PY, clone 4G10) and anti-p130cas mAb (Transduction Labs) were used for immunoblot. (FIG. 5a). Next, immunoprecipitation was performed on pre-cleared lysate with anti-CrkII Ab (rabbit polyclonal, Santa Cruz). Protein A-agarose beads were used to isolate CrkII and associating proteins. SDS-PAGE was performed and blotted with anti-PY and anti-CrkII antibodies (b). 293T cells were also incubated with apoptotic T cells at a ratio of 1:10 and after 30 minutes lysed as described above. Total lysate (c) or CrkII immunoprecipitated protein complexes were run on an SDS-PAGE (d) and blotted with anti-PY(c, d). C, media control. AC, apoptotic cells.

Cells plated onto poly-L-lysine coated plates which promotes a non-integrin mediated charge dependant cell adhesion did not induce p130cas phosphorylation. As a positive control, cells were plated on fibronectin (FN), which triggers the αᵥβ₁ heterodimer and p130cas tyrosine phosphorylation via the recruitment of FAK58. Importantly, phosphorylation of p130cas after 293T cells were co-cultured with apoptotic cells was observed (FIGS. 5c,d). With respect to the formation of focal adhesions, it has been reported that p130ocas efficiently recruits CrkII, an adaptor protein consisting of one SH2 (Src homology 2) and two SH3 domain. It has also been shown that the SH3 domain of CrkII binds proteins including c-abl, C3G and DOCK180 (50, 51). To demonstrate a link between β₅ activation and the recruitment of CrkII, Crk binding proteins were co-immunoprecipitated from the 293T cell lysates and samples were probed with the anti-phosphotyrosine specific mAb PY20 (FIGS. 5b,d). As shown, several inducible Crk-associated proteins were observed following activation of the β₅ receptor, some with molecular weights not typical of known Crk-associated proteins (FIG. 5b). The amount of CrkII-associated phosphorylated p130cas was increased significantly in cells which had been exposed to VN (FIG. 5b). Additionally, increased phosphorylation of CrkII at the Y222 position was demonstrated (FIG. 5b) as well as in increased CrkII-association with tyrosine phosphorylated DOCK180 (FIG. 5b). This data directly implicates a p130cas/CrkII/DOCK180 complex in the β₅-mediated phagocytosis of apoptotic cells. This result offers a functional link to the phagocytosis of apoptotic cells in C. elegans. DOCK180, originally cloned based on its interaction with the SH3 domain of CrkII61, is the human homolog of CED-562, which acts in a pathway which includes CED-2 and CED-1063. CED-2 is the cellular homolog of c-Crk II, which supports the data herein that the pathways important for phagocytosis of apoptotic cells are conserved from worms to humans.

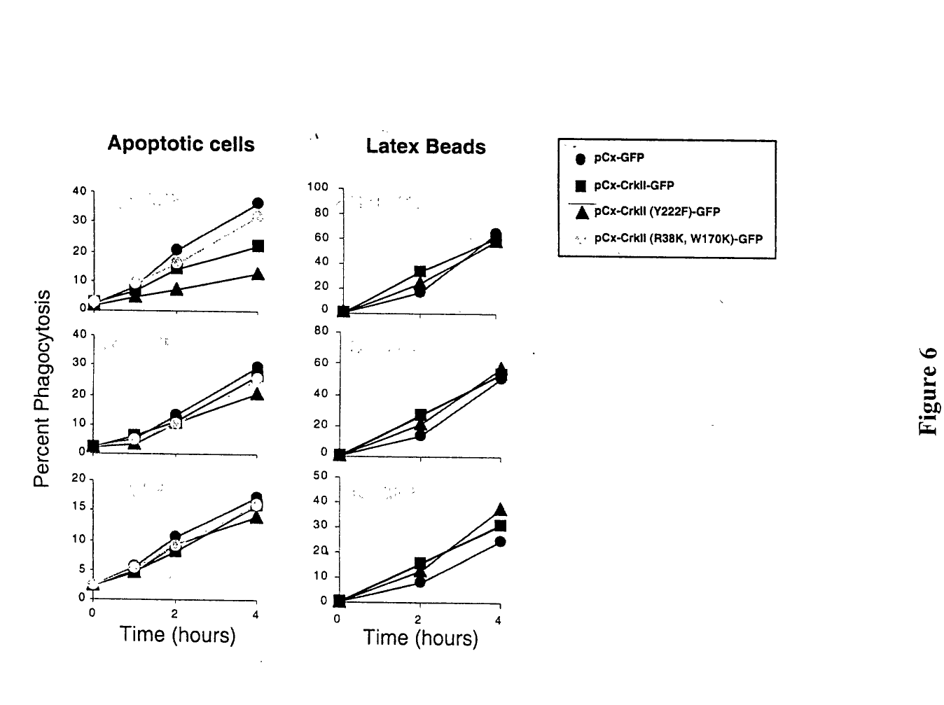

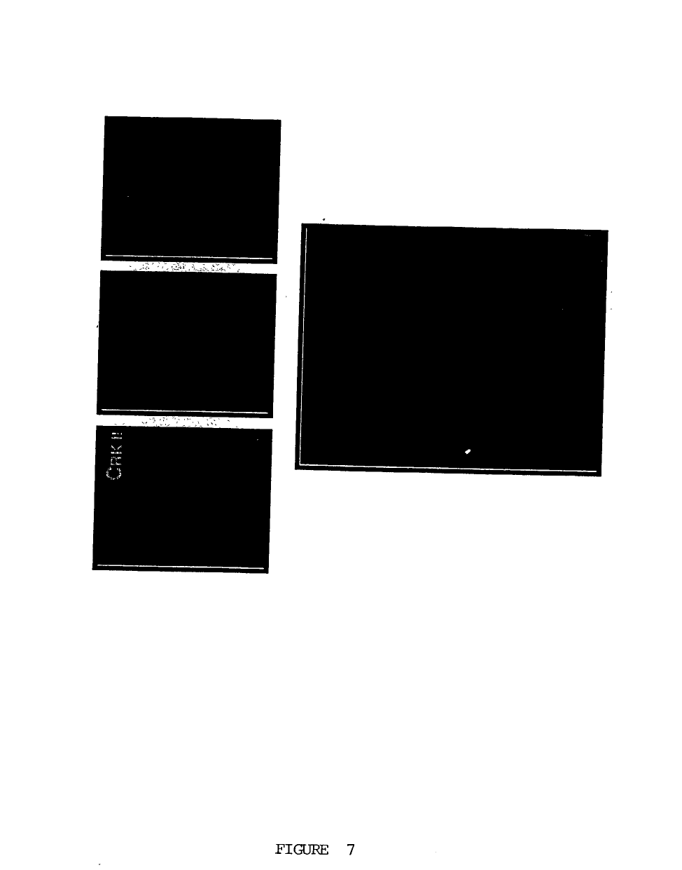

To confirm that CrkII and DOCK180 are involved in the phagocytosis of apoptotic cell in human cells, the FACS based phagocytosis assay was employed. Mutant and wild type CrkII and DOCK180 were cloned into bicistronic vectors for use in 293T cell transfections. High expression levels of Y222F Crk (a dominant negative Crk which can not be phosphorylated) were found to inhibit the uptake of apoptotic cells by the 293T cells. Surprisingly and unexpectedly, the overexpression of wild-type CrkII also inhibited the engulfinent of apoptotic cells. This result is understood to reflect the sequestration of DOCK180 or other relevant molecules in the cytosol, thus preventing the recruitment of the necessary scaffolding proteins to the plasma membrane for actin cytoskeletal rearrangement. This interpretation is supported by the overexpression of the CrkII double mutant which has point mutations that disrupt both SH2 and SH3 interaction (51). This mutant is essentially a non-functional Crk and as expected, does not effect the phagocytosis of apoptotic cells (FIG. 6a).

To demonstrate that inhibition is specific to the uptake of apoptotic cells, a control particle was used in the phagocytosis experiments (52). Red fluorescent latex beads were co-cultured with 293T cells expressing the various CrkII constructs and phagocytosis was measured by FACS®. (Note, latex beads are phagocytosed by scavenger receptors, and hence an index of non-specific engulfment). No inhibition was observed with either Y222F CrkII or wild-type CrkII indicating that the effect we are observing is restricted to the uptake of apoptotic cells (FIG. 6b).

Similar experiments were preformed using mutant DOCK180 constructs which lacked the CrkII consensus sequence. Results obtained were consistent with the CrkII data, as overexpression of mutant and native DOCK180 inhibited phagocytosis. DOCK180 may interact with Rac1, thus suggesting that sequestration of this effector molecule in the cytosol blocks uptake. Taken together, these data suggest that phagocytosis is a dynamic molecular process—dominant-negative, as well as wild-type CrkII and DOCK180 overexpression are inhibitory. These data are consistent with the previous findings that CrkII has both positive and negative effects on cell adhesion, reflecting a balance between inductive and destabilizing forces on the actin cytoskeleton.

### EXAMPLE 4

### PTKs and CrkII are Employed by DCs for the Uptake of Apoptotic Cells

To address the importance of protein tyrosine kinases (PTKs) in β₅-mediated phagocytosis by human DCs, and demonstrate the relevance of our findings in 293T cells to DC biology, cell permeable tyrosine kinase inhibitors Herbimycin A and Lavendustin (Gibco BRL) were utilized. Interestingly, phagocytosis was inhibited by 50-60%. This level of inhibition was equivalent to that observed when using mAbs specific for β₅. Furthermore, when DCs were co-cultured with apoptotic cells in the presence of both tyrosine kinase inhibitors and anti-β₅ Abs, no additional uptake was observed. These data are consistent with a role for tyrosine kinase activation following β₅ engagement.

To establish a link between Crk and the β₅ pathway, indirect immunofluorescence was performed to detect the localization of Crk within the DC, post-engulfment of an apoptotic cell. Human DCs were co-cultured with influenza-infected apoptotic cells and immunofluorescence was performed. The apoptotic cell (identified by labeling with anti-influenza nucleoprotein antibody) could be detected within the cytoplasm of the DCs. Notably, the limiting membrane of the phagosome is enriched for CrkII (FIG. 7). In detail, influenza infected RAW cells were induced to undergo apoptosis using 120 mJ/cm⁻2 UVB, and after 8 hours, immature DCs were added. Co-cultures were placed on a poly-lysine coverslip after 1 hour and fixed with 4% para-formaldehyde. Intracellular staining was performed using anti-CrkII antibody (Santa Cruz Biotech.) followed by FITC-conjugated Goat anti-rabbit secondary antibody (Jackson Immunochemicals); and anti-influenza nucleoprotein (NP) antibody (clone HB65, ATCC) followed by Texas Red conjugated Goat anti-mouse secondary antibody. Finally, cells were labeled with DAPI to identify the nuclei of cells (Sigma Chemicals).

The foregoing work establishes that (i) integrins act as signaling receptors, and not simply as adhesion molecules, directly facilitating internalization of the dying cell; (ii) the p130cas-Crk-DOCK180 molecular complex is involved in the phagocytosis of apoptotic cells in humans; (iii) phagocytosis is a dynamic process and that the relative expression of signaling molecules must be balanced.

### EXAMPLE 5

### Signaling via the β₅ Integrin Mediates DCs Phagocytosis of Apoptotic Cells

### Preliminary Studies of Primary Human DCs

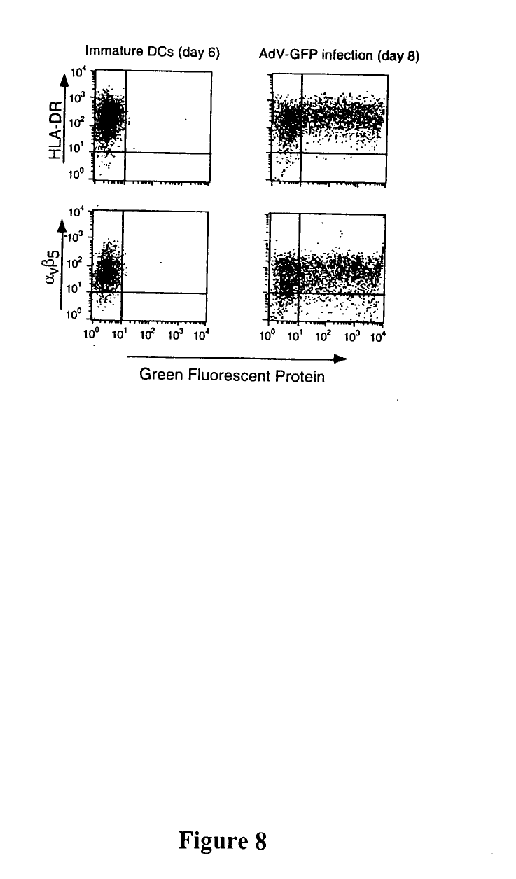

AdV-GFP has been shown capable of transducing DCs at an efficiency of 60-90%, without altering the maturation state of the cells, based on moderate expression of HLA-DR, a maturation marker present on human Dcs. Immature DCs were phenotyped by FACS® analysis on Day 6 (FIG. 8a), and infected with an adenovirus expressing GFP. After 40 hours, DCs were analyzed for expression levels of HLA-DR and β₅ (FIG. 8b). This is consistent with the findings of others who have shown that adenoviral infection does not trigger DC maturation (52).This latter point concerning the maturation state of the DC is important, as it is the immature DC which preferentially captures apoptotic cells (8). An obvious concern is the fact that AdV utilizes the β₅ receptor for entry into DCs and might alter expression level of this receptor (53). Of note, the viral vectors being employed are replication incompetent, and it was hoped that after 36-48 hrs, there would be equivalent levels of β₅ expressed by the DC. This was evaluated by FACS® analysis and in fact, the β₅ expression was unchanged as compared to uninfected DCs (FIG. 8), thus permitting the use of AdV for gene delivery to immature primary DCs. Phagocytosis experiments carried out using AdV-GFP demonstrated comparable levels of uptake compared to uninfected controls.

Recombinant AdV vectors expressing wild type β₅ and β₅ΔC may be prepared using the well-characterized AdEasy homologous recombination strategy described by Vogelstein and colleagues (54). To construct AdV expression constructs, the gene of interest (e.g. β₅ integrin receptor subunit) is cloned into a shuttle vector (pAdTract-CMV) with the following modifications to express β₅ and GFP from a bicistronic transcript using an internal ribosomal entry site (IRES) to drive the expression of the GFP. Hence, the gene is expressed as has been done in the pCX retroviral gene-β₅ integrin construct. The pAdTract-CMV constructs are co-transformed with supercoiled adenovirus vector (pAdEasy) into the E. coli strain BJ5183, and recombination between the two plasmids is selected based on the combined kanomycin and ampicillin resistance. Plasmid DNA is isolated and transfected into a packaging line (293 cells) and virus is harvested 7 to 10 days later. This strategy has been utilized to propagate GFP-expressing virus, and for generating recombinant wild type β₅-GFP and β₅ΔC-GFP expressing constructs. Once high titer virus expressing β₅ integrin constructs is prepared, immature DCs are infected and FACS®-based phagocytosis assays are preformed. In all experiments, primary human immature DCs are prepared from peripheral blood precursors using GM-CSF and IL-4 (55, 56). Peripheral blood mononuclear cells (PBMCs) can be isolated from leukopaks, which are commercially available, for example, at the Red Cross Blood Center. The DCs prepared are infected with the various AdV vectors (10⁶ cells will be used per group), and plated in 48 well plates at a cell dose of 10⁵/well. The DCs are incubated for 36-48 hours, allowing for β5and β5ΔC expression, at which time PHK26 labeled apoptotic T cells are added to the cultures. At various time points, individual wells are resuspended and analyzed by FACS for their having captured an apoptotic cell. β₅ expression levels is assessed throughout the experiments to show that (i) overexpression of integrin receptors do not effect DC maturation; (ii) that the AdV delivery system does not interfere with 5 expression; and (iii) that the observation holds true in DCs that β5 overexpression results in the increased expression of the v integrin.

### EXAMPLE 6

### Importance of the β₅ Integrin Receptor in the Cross-Presentation of Antigen

By analyzing the effect on (i) the generation of defined MHC I/peptide complexes, and (ii) the activation of antigen-specific CTLs in an overexpressing β5 mutant that disrupts uptake of apoptotic cells via the β₅ receptor, the importance of the β5 integrin receptor is demonstrated. Using the constructs and gene delivery systems outlined above, wild-type and mutant β5 gene products are expressed in DCs. These DCs are co-cultured with influenza infected apoptotic cells at various ratios and after 6, 12 and 24 hours, the DCs are analyzed for the expression of a defined MHC I/peptide complex, by employing an antibody specific for a shared epitope in the MHC I/matrix peptide complex. The matrix peptide, GILGFVFTL, is the HLA-A2.1 restricted immunodominant epitope derived from the influenza matrix protein (57, 58). Given the requirement for this specific MHC allele, DCs derived from HLA-A2.1 individuals are employed. Use of a bicistronic expression system permit correlating overexpression of β5 and β5ΔC with cross-presentation by the DCs. These experiments are supported by the previously demonstration that murine DCs efficiently cross-present an epitope derived from exogenous I-E protein on MHC II (59). Detection of this complex was measured using an antibody specific for the E peptide (residues 56-73) presented on I-Ab. Using ¹²⁵I-labeled 14-4-4S mAb (anti-I-E), it was possible to determine the number of E molecules present in the apoptotic cells, demonstrating that cross-presentation of apoptotic cells is one to ten thousand times more efficient in generating MHC class II/peptide complexes than preprocessed I-E peptide (73).

### EXAMPLE 7

### Kinetics of Phagocytosis in 293T Cells

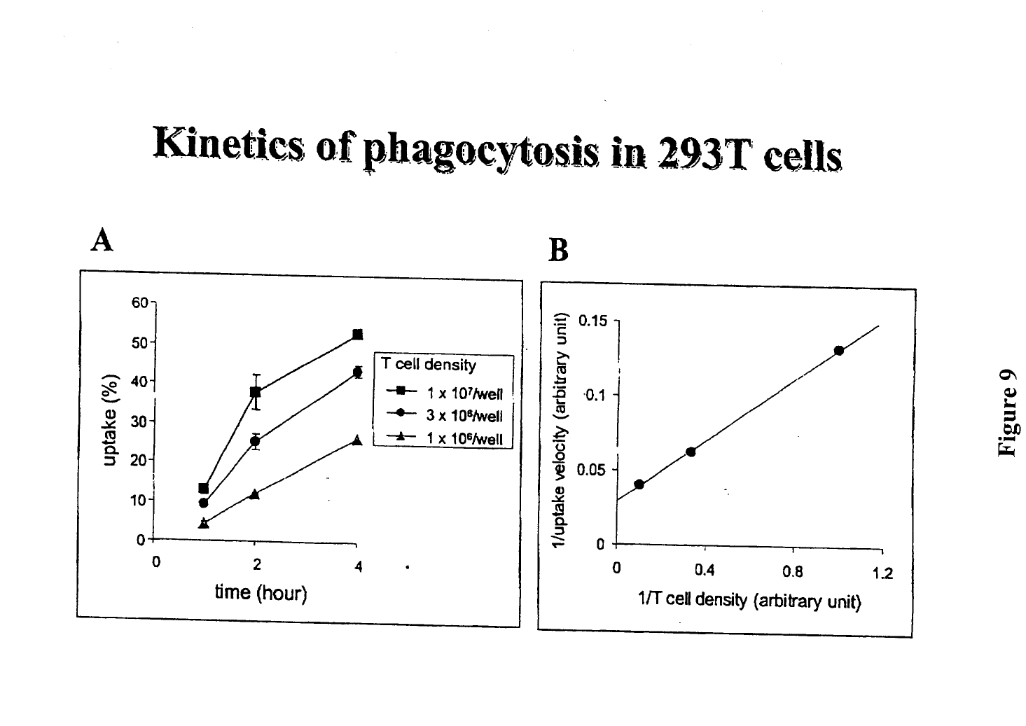

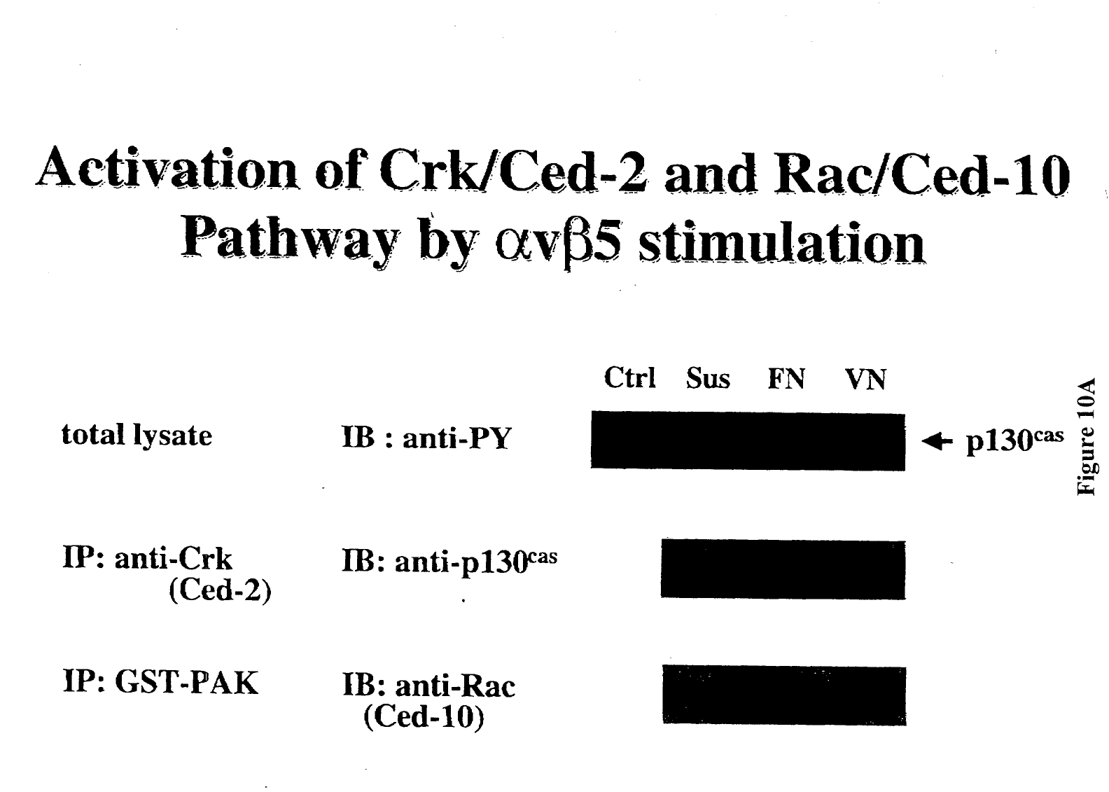

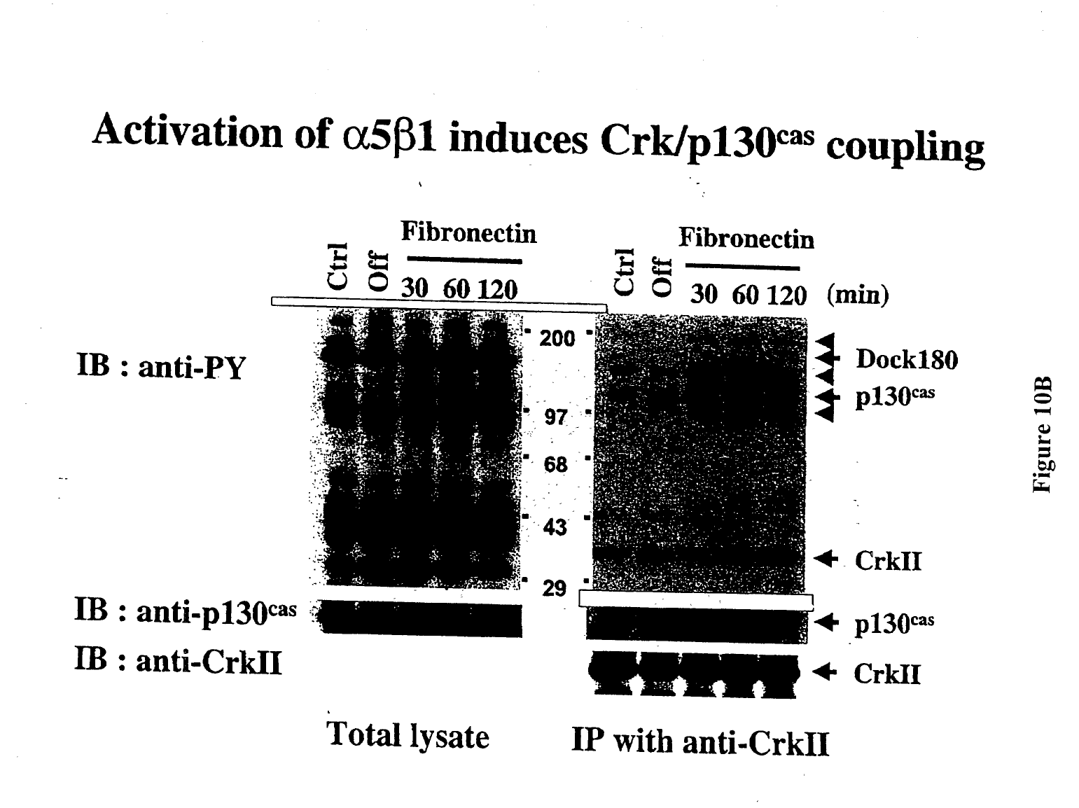

The phagocytosis of apoptotic T cells by 293T cells was monitored as a function of T cell number (FIG. 9). In FIG. 9B, a double reciprocal plot of 1/Velocity versus 1/T cell density is shown. Note that the plot is linear indicating the kinetics follow a Michaelis Menten pattern. This is important as it indicates the phagocytosis is saturatable, consistent of a receptor-mediated process.

Various publications are cited herein, the disclosures of which are incorporated by reference in their entireties.

1. Heemels, M. T. and H. Ploegh, Generation, translocation, and presentation of MHC class I-restricted peptides. Annu Rev Biochem, 1995. 64: p. 463-91.

2. Pamer, E. and P. Cresswell, Mechanisms of MHC class I-restricted antigen processing. Annu Rev Immunol, 1998. 16: p. 323-58.

3. Bevan, M. J., Cross-priming for a secondary cytotoxic response to minor H antigens with H-2 congeneic cells which do not cross-react in the cytotoxic assay. J. Exp Med, 1976. 143(5): p. 1283-8.

4. Bevan, M. J., Minor H antigens introduced on H-2 different stimulating cells cross-react at the cytotoxic T cell level during in vivo priming. J. Immunol, 1976. 117(6): p. 2233-8.

5. Huang, A. Y., et al., Role of bone marrow-derived cells in presenting MHC class I-restricted tumor antigens. Science, 1994. 264(5161): p. 961-5.

6. Sigal, L. J., et al., Cytotoxic T-cell immunity to virus-infected non-haematopoietic cells requires presentation of exogenous antigen. Nature, 1999. 398(6722): p. 77-80.

7. Albert, M. L., B. Sauter, and N. Bhardwaj, Dendritic cells acquire antigen from apoptotic cells and induce class I-restricted CTLs. Nature, 1998. 392(6671): p. 86-9.

8. Albert, M. L., et al., Immature dendritic cells phagocytose apoptotic cells via alphavbeta5 and CD36, and cross-present antigens to cytotoxic T lymphocytes. J Exp Med, 1998. 188(7): p. 1359-68.

9. Albert, M. L. and N. Bhardwaj, Resurrecting the Dead: DCs cross-present antigen derived from apoptotic cells on MHC I. The Immunologist, 1998. 6: p. 194-199.

10. Savill, J., Recognition and phagocytosis of cells undergoing apoptosis. Br Med Bull, 1997. 53(3): p. 491-508.

11. Fadok, V. A. and P. M. Henson, Apoptosis: getting rid of the bodies. Curr Biol, 1998. 8(19): p. R693-5.

12. Savill, J., Apoptosis. Phagocytic docking without shocking [news; comment]. Nature, 1998. 392(6675): p. 442-3.

13. Platt, N., R. P. da Silva, and S. Gordon, Recognizing death: the phagocytosis of apoptotic cells. Trends Cell Biol, 1998. 8(9): p. 365-72.

14. Albert, M. L., et al., Uptake and Presentation of Phgocytosed Antigens by Dendritic Cells, in Phagocytosis and Pathogens, S. Gordon, Editor. 1999: Philadelphia. p. 363-378.

15. Albert et al., 1998, J. Exp. Med. 1121.

16. Voll, R. E., et al., Immunosuppressive effects of apoptotic cells [letter]. Nature, 1997. 390(6658): p. 350-1.

17. Fadok, V. A., et al., Macrophages that have ingested apoptotic cells in vitro inhibit proinflammatory cytokine production through autocrine/paracrine mechanisms involving TGF-beta, PGE2, and PAF. J Clin Invest, 1998. 101(4): p. 890-8.

18. Steinman, R. M., et al., The Induction of Tolerance by Dendritic Cells That Have Captured Apoptotic Cells. J Exp Med, 2000. 191: p. 411-416.

19. Romani, N., et al., Presentation of exogenous protein antigens by dendritic cells to T cell clones. Intact protein is presented best by immature, epidermal Langerhans cells. J Exp Med, 1989. 169(3): p. 1169-78.

20. Banchereau, J. and R. M. Steinman, Dendritic cells and the control of immunity. Nature, 1998. 392(6673): p. 245-52.

21. Albert, M. L., et al., Tumor-specific killer cells in paraneoplastic cerebellar degeneration. Nat Med, 1998. 4(11): p. 1321-4.

22. Randolph, G. J., et al., Differentiation of monocytes into dendritic cells in a model of transendothelial trafficking. Science, 1998. 282(5388): p. 480-3.

23. Larsen, C. P., et al., Migration and maturation of Langerhans cells in skin transplants and explants. J Exp Med, 1990. 172(5): p. 1483-93.

24. Cohen, G. M., Biochen, J. (1997) 326:1-16.

25. Miller, D. K. 1997 Sem. Immunol 5:35-49.

26. Wyllie, A. H. 1997 Eur. J. Cell. Biol. 73:189-197.

27. Van Der Brogen, P., C. Traverari, P. Chomez, C. Lurquin, E. De Plaen, B. Van Deneynde, A. Knuth, and T. Boon, 1991, Science 254:1643-1647.

28. Young, J. W. and R. M. Steinman, 1990, J. Exp. Med., 171:1315-1332.

29. McHichael, A. J. and B. A. Askonas, 1978, Eur. J. Immunol., 8:705-711.

30. Koup, R. A., C. A. Pikora, K. Luzuriaga, D. B. Brettler, E. S. Day, G. P. Mazzara, and J. L. Sullivan, 1991 J. Exp. Med. 174:1593-1600.

31. Carmichael, A, X. Jin, P. Sissons, and L. Borysiewicz, 1993, J. Exp. Med. 177:249-256.

32. Johnson, R. P., A. Trocha, T. M. Buchanan and B. D. Walker, 1992, J. Exp. Med. 175:961-971.

33. Hill, A. V. S., J. Elvin, A. C. Willis, M. Aidoo, C. E. M. Allsopp, F. M. Gotch, X. M. Gao, M., Takiguchi, B. M. Greenwood, A. R. M. Townsend, A. J. McHichael, and H. C. Whittle, 1992, Nature 360:434-439.

34. Rovere, P., et al., Dendritic cells preferentially internalize apoptotic cells opsonized by anti-beta2-glycoprotein I antibodies [In Process Citation]. J Autoimmun, 1998. 11(5): p. 403-11.

35. Platt, N., et al., Role for the class A macrophage scavenger receptor in the phagocytosis of apoptotic thymocytes in vitro. Proc Natl Acad Sci USA, 1996. 93(22): p. 12456-60.

36. Mevorach, D., et al., Complement-dependent clearance of apoptotic cells by human macrophages [In Process Citation]. J Exp Med, 1998. 188(12): p. 2313-20.

37. Fadok, V. A., et al., Exposure of phosphatidylserine on the surface of apoptotic lymphocytes triggers specific recognition and removal by macrophages. J Immunol, 1992. 148(7): p. 2207-16.

38. Savill, J., Phagocyte recognition of apoptotic cells. Biochem Soc Trans, 1996. 24(4): p. 1065-9.

39. Sutterwala, F. S., et al., Selective suppression of interleukin-12 induction after macrophage receptor ligation. J Exp Med, 1997. 185(11): p. 1977-85.

40. Yoshida, Y., et al., Monocyte induction of L-10 and down-regulation of IL-12 by iC3b deposited in ultraviolet-exposed human skin. J Immunol, 1998. 161: p. 5873-9.

41. Hughes, J., et al., Human glomerular mesangial cell phagocytosis of apoptotic neutrophils: mediation by a novel CD36-independent vitronectin receptor/thrombospondin recognition mechanism that is uncoupled from chemokine secretion. J Immunol, 1997. 158(9): p. 4389-97.

42. Savill, J., et al., Thrombospondin cooperates with CD36 and the vitronectin receptor in macrophage recognition of neutrophils undergoing apoptosis. J Clin Invest, 1992. 90(4): p. 1513-22.

43. Ren, Y., et al., CD36 gene transfer confers capacity for phagocytosis of cells undergoing apoptosis. J Exp Med, 1995. 181(5): p. 1857-62.

44. Finnemann, S. C., et al., Phagocytosis of rod outer segments by retinal pigment epithelial cells requires alpha(v)beta5 integrin for binding but not for internalization. Proc Natl Acad Sci USA, 1997. 94(24): p. 12932-7.

45. Sparrow, J. R., et al., CD36 expression is altered in retinal pigment epithelial cells of the RCS rat. Exp Eye Res, 1997. 64(1): p. 45-56.

46. McLean, J. W., et al., cDNA sequence of the human integrin beta 5 subunit. J Biol Chem, 1990. 265(28): p. 17126-31.

47. Savill, J., et al., Vitronectin receptor-mediated phagocytosis of cells undergoing apoptosis. Nature, 1990. 343(6254): p. 170-3.

48. Clark, E. A. and J. S. Brugge, Integrins and signal transduction pathways: the road taken. Science, 1995. 268(5208): p. 233-9.

49. Wayner, E. A., R. A. Orlando, and D. A. Cheresh, Integrins alpha v beta 3 and alpha v beta 5 contribute to cell attachment to vitronectin but differentially distribute on the cell surface. J Cell Biol, 1991.113: p. 919-29.

50. Tanaka, S., et al., Both the SH2 and SH3 domains of human CRK protein are required for neuronal differentiation of PC12 cells. Mol Cell Biol, 1993. 13: p. 4409-15.

51. Birge, R. B., et al., SH2 and SH3-containing adaptor proteins: redundant or independent mediators of intracellular signal transduction. Genes Cells, 1996. 1(7): p. 595-613.

52. Zhong, L., et al., Recombinant adenovirus is an efficient and non-perturbing genetic vector for human dendritic cells. Eur J Immunol, 1999. 29: p. 964-72.

53. Nemerow, G. R. and P. L. Stewart, Role of alpha(v) integrins in adenovirus cell entry and gene delivery. Microbiol Mol Biol Rev, 1999. 63: p. 725-34.

54. He, T. C., et al., A simplified system for generating recombinant adenoviruses. Proc Natl Acad Sci USA, 1998. 95: p. 2509-14.

55. Bender, A., et al., Improved methods for the generation of dendritic cells from nonproliferating progenitors in human blood. J Immunol Methods, 1996. 196(2): p. 121-35.

56. Romani, N., et al., Generation of mature dendritic cells from human blood. An improved method with special regard to clinical applicability. J Immunol Methods, 1996. 196(2): p. 137-51.

57. Gotch, F., et al., Identification of viral molecules recognized by influenza-specific human cytotoxic T lymphocytes. J Exp Med, 1987. 165(2): p. 408-16.

58. Gotch, F., et al., Cytotoxic T lymphocytes recognize a fragment of influenza virus matrix protein in association with HLA-A2. Nature, 1987. 326(6116): p. 881-2.

59. Inaba, K., et al., Efficient presentation of phagocytosed cellular fragments on the major histocompatibility complex class II products of dendritic cells [In Process Citation]. J Exp Med, 1998. 188(11): p. 2163-73.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

What is claimed is:

1. A method for enhancing the ability of a phagocyte to capture an apoptotic-cell-delivered antigen comprising genetically modifying said phagocyte to
i) express an apoptotic cell receptor with enhanced ability to capture apoptotic cells; or
ii) increase expression of an apoptotic-cell receptor.

2. The method of claim 1 wherein said phagocyte is a professional phagocyte.

3. The method of claim 2 wherein said professional phagocyte is an antigen presenting cell.

4. The method of claim 3 wherein said antigen presenting cell is a dendritic cell.

5. The method of claim 4 wherein said dendritic cell is a myeloid dendritic cell or a lymphoid dendritic cell.

6. The method of claim 3 wherein said antigen presenting cell is a macrophage.

7. The method of claim 3 wherein said antigen presenting cell is a B cell

8. The method of claim 2 wherein said professional phagocyte is a neutrophil.

9. The method of claim 1 wherein said phagocyte is a nonprofessional phagocyte.

10. The method of claim 1 wherein said nonprofessional phagocyte is a keratinocyte, a fibroblast, an epithelial cell or an endothelial cell.

11. The method of claim 1 wherein said phagocyte is a human phagocyte.

12. The method of claim 1 wherein said phagocyte is a non-human phagocyte.

13. The method of claim 1 wherein said apoptotic-cell receptor is selected from the group consisting of a member of the Fc receptor family, a member of the scavenger receptor family, CD14, a member of the ABC-1 family of transporters, a member of the C-type lectin family, an integrin receptor β subunit other than β₁, an integrin heterodimer other than that comprising β₁, an integrin heterodimer comprising a chimeric β subunit other than β₁, and an integrin heterodimer comprising a mutant β subunit.

14. The method of claim 1 wherein said integrin β subunit is β₅.

15. The method of claim 13 wherein said integrin heterodimer is αᵥβ₅.

16. The method of claim 13 wherein said integrin receptor heterodimer comprising a chimeric subunit comprises a wild-type α subunit and a chimeric β subunit, wherein the chimeric β subunit comprises an extracellular β₅ domain fused with a signaling domain derived from a molecule selected from the group consisting of an integrin β subunit other than β₁, a member of the Fc receptor family, a member of the scavenger receptor family, and a member of the C-type lectin family.

17. The method of claim 16 wherein said signaling domain derived from a member of the Fc receptor family is the FcγRI, FcγRIIA, FcγRIIB, or FcγRIII α-chain.

18. The method of claim 16 wherein said signaling domain derived from an integrin β subunit other than β₁ is that of β₂, β₃ or β₅.

19. The method of claim 1 wherein said genetically modifying said phagocyte is carried out by a method selected from the group consisting of transfection and gene transfer.

20. The method of claim 19 wherein said transfection is performed using a viral vector.

21. The method of claim 19 wherein said transfection is performed by a plasmid.

22. The method of claim 19 wherein said transfection is performed by microinjection.

23. The method of claim 19 wherein said transfection is performed using a gene gun.

24. A method for enhancing the capture of an apoptotic-cell-delivered antigen by a phagocyte comprising the steps of
(a) providing a phagocytic cell of claim 1; and
(b) exposing said genetically-modified phagocyte to an apoptotic cell comprising an antigen.

25. The method of claim 24 wherein said phagocytic cell is capable of cross-presenting said antigen.

26. A genetically-modified phagocyte with enhanced ability to capture an apoptotic-cell-delivered antigen, said genetically modified phagocyte prepared by genetically modifying said phagocyte to increase expression of an apoptotic-cell receptor in accordance with claim 1.

27. A method for enhancing the ability of a dendritic cell or precursor thereof to cross-present an apoptotic-cell-delivered antigen comprising genetically modifying said dendritic cell to increase expression of an apoptotic-cell receptor capable of cross-presenting said antigen.

28. The method of claim 27 wherein said dendritic cell is a myeloid dendritic cell.

29. The method of claim 27 wherein said dendritic cell is a lymphoid dendritic cell.

30. The method of claim 27 wherein said apoptotic-cell receptor is selected from the group consisting of a member of the Fc receptor family, a member of the scavenger receptor family, a member of the ABC-1 family of transporters, a member of the C-type lectin family, an integrin receptor β subunit other than β₁, an integrin receptor heterodimer comprising a β subunit other than β₁, an integrin heterodimer comprising a chimeric β subunit other than β₁, and an integrin heterodimer comprising a mutant β subunit.

31. The method of claim 30 wherein said integrin β subunit is β₅.

32. The method of claim 30 wherein said integrin heterodimer is αᵥβ₅.

33. The method of claim 30 wherein said integrin heterodimer comprising a chimeric β subunit comprises a wild-type α subunit and a chimeric β subunit, wherein the chimeric β subunit comprises an extracellular β₅ domain fused with a signaling domain derived from a molecule selected from the group consisting of an integrin β subunit other than β₁, a member of the Fc receptor family, a member of the scavenger receptor family, and a member of the C-type lectin family.

34. The method of claim 33 wherein said signaling domain derived from a member of the Fc receptor family is FcRγI, FcγRIIA, FcγRIIB, or FcRγIII α-chain.

35. The method of claim 33 wherein said signaling domain derived from an integrin β subunit other than β₁ is that of β₂, β₃ or β₅.

36. The method of claim 27 wherein said genetically modifying said dendritic cell or precursor thereof is carried out by a method selected from the group consisting of transfection and gene transfer.

37. The method of claim 36 wherein said transfection is performed using a viral vector.

38. The method of claim 36 wherein said transfection is performed by a plasmid.

39. The method of claim 36 wherein said transfection is performed by microinjection.

40. The method of claim 36 wherein said transfection is performed using a gene gun.

41. A method for enhancing the ability of a phagocyte other than a dendritic cell to capture and degrade an apoptotic-cell-delivered antigen comprising genetically modifying said phagocyte to increase expression of an apoptotic-cell receptor.

42. The method of claim 41 wherein said phagocyte is a professional phagocyte.

43. The method of claim 42 wherein said professional phagocyte is an antigen presenting cell.

44. The method of claim 43 wherein said antigen presenting cell is a macrophage.

45. The method of claim 41 wherein said phagocyte is a nonprofessional phagocyte.

46. The method of claim 41 wherein said nonprofessional phagocyte is a keratinocyte, a fibroblast, an epithelial cell or an endothelial cell.

47. The method of claim 41 wherein said phagocyte is a human phagocyte.

48. The method of claim 41 wherein said phagocyte is a non-human phagocyte.

49. The method of claim 41 wherein said apoptotic-cell receptor is selected from the group consisting of a member of the Fc receptor family, a member of the scavenger receptor family, CD14, a member of the ABC-1 family of transporters, a member of the C-type lectin family, an integrin β subunit other than β₁, an integrin heterodimer comprising a β subunit other than β₁, an integrin heterodimer comprising a chimeric subunit other than β1, and an integrin heterodimer comprising a mutant β subunit.

50. The method of claim 49 wherein said integrin β subunit is β₅.

51. The method of claim 49 wherein said integrin heterodimer is αᵥβ₅.

52. The method of claim 49 wherein said integrin heterodimer comprising a chimeric β subunit comprises a wild-type α subunit and a chimeric β subunit, wherein the chimeric β subunit comprises an extracellular β₅ domain fused with a signaling domain derived from a molecule selected from the group consisting of an integrin β subunit other than β₁, a member of the Fc receptor family, a member of the scavenger receptor family, and a member of the C-type lectin family.

53. The method of claim 52 wherein said signaling domain derived from a member of the Fc receptor family is FcRγI, FcγRIIA, FcγRIIB, or FcRγIII α-chain.

54. The method of claim 52 wherein said signaling domain derived from an integrin β subunit other than β₁ is that of β₂, β₃ or β₅.

55. The method of claim 41 wherein said genetically modifying said phagocyte is carried out by a method selected from the group consisting of transfection and gene transfer.

56. The method of claim 55 wherein said transfection is performed using a viral vector.

57. The method of claim 55 wherein said transfection is performed by a plasmid.

58. The method of claim 55 wherein said transfection is performed by microinjection.

59. The method of claim 55 wherein said transfection is performed using a gene gun.

60. A method for enhancing the ability of a dendritic cell or precursor thereof to capture and degrade an apoptotic-cell-delivered antigen comprising genetically modifying said dendritic cell or precursor thereof to increase expression of an apoptotic-cell receptor comprising an integrin heterodimer comprising an αᵥ subunit and a β₁ or β₃ subunit, or a chimeric β subunit with a β₁ or CD14 signaling domain.

61. A method for enhancing cross-priming of T cells by dendritic cells using an apoptotic-cell-delivered antigen comprising the steps of
(a) genetically modifying said dendritic cells or precursors thereof to increase expression of an apoptotic-cell receptor capable of promoting capture of apoptotic cells and enhancing cross-priming of T cells; and
(b) exposing said genetically-modified dendritic cells to an apoptotic cell comprising an antigen in the presence of at least one immunostimulatory exogenous factor or antigen-specific CD4 helper T cells;
wherein said dendritic cells have enhanced ability promote the formation of antigen-specific CD8 cells.

62. The method of claim 61 wherein said apoptotic-cell receptor capable of promoting cross-priming of T cells is selected from the group consisting of a cross-priming promoting member of the Fc receptor family, a member of the scavenger receptor family, a member of the C-type lectin family, a β integrin receptor subunit other than β₁, an integrin receptor heterodimer other than that comprising β₁, an integrin heterodimer comprising a chimeric β subunit other than β₁, and an integrin heterodimer comprising a mutant β subunit.

63. The method of claim 62 wherein said integrin β subunit is β₅.

64. The method of claim 62 wherein said integrin heterodimer is αᵥβ₅.

65. The method of claim 62 wherein said integrin heterodimer or β subunit comprises a chimeric β subunit with an extracellular β₅ domain and an signaling domain selected from the group consisting of integrin β₂, integrin β₃, integrin β₅, FcgRI α-chain, FcgIIA α-chain or FcgRIII α-chain.

66. The method of claim 62 wherein said dendritic cells are myeloid dendritic cells.

67. The method of claim 62 wherein said dendritic cells are lymphoid myeloid dendritic cells.

68. The method of claim 62 wherein said antigen is a tumor antigen and said T cells are tumor-specific T cells.

69. The method of claim 62 wherein said antigen is a viral antigen and said T cells are virus-specific or virally-infected cell specific T cells.

70. The method of claim 62 wherein said enhanced cross-priming of T cells with said antigen results in enhanced killing of tumors or virus-infected cells

71. The method of claim 62 wherein said enhanced cross-priming of T cell results in the enhanced formation of antigen-specific CD4 helper cells.

72. The method of claim 62 wherein said immunostimulatory exogenous factor is at least one of CD40 ligand, TRANCE, TRAIL, OX40 or an alternate member of the TNF superfamily, or thalidomide.

73. The method of claim 72 wherein said member of the TNF superfamily is TRAIL.

74. A method for enhancing cross-tolerance of T cells to an apoptotic-cell-delivered antigen by dendritic cells or precursors thereof comprising the steps of
(a) genetically modifying said dendritic cells or precursors thereof to increase expression of an apoptotic-cell receptor capable of promoting capture of apoptotic cells and enhancing cross-tolerance of T cells; and
(b) exposing said genetically-modified phagocyte to an apoptotic cell comprising an antigen in the presence of immunosuppressive exogenous factors or in the absence of the combination of antigen-specific CD4 helper T cells and immunostimulatory exogenous factors;
wherein said dendritic cells have increased ability tolerize antigen-specific CD8 cells.

75. The method of claim 74 wherein said apoptotic-cell receptor capable of enhancing cross-tolerance of T cells is an integrin heterodimer with a β2 subunit, a member of the Fc receptor family, or a chimeric β subunit with an extracellular β₅ domain and an signaling domain selected from the group consisting of integrin β₂ or FcγRIIB α-chain.

76. The method of claim 74 wherein said immunosuppressive exogenous factor is at least one of TGF-β, IL-10, IL-4, IL-5, IL-13, FK506 or an agent that binds to FKBP12.

77. The method of claim 74 wherein said cross-tolerance results in a decrease in autoreactive T cells to said antigen.

78. A method for treating an autoimmune disease comprising carrying out the method of claim 74.

79. The method of claim 77 wherein said autoimmune disease is psoriasis, Crohn's disease, rheumatoid arthritis, or multiple sclerosis.

80. A method for reducing the immune response to a transplant antigen comprising carrying out the method of claim 74, wherein said antigen is an allogeneic transplant antigen or a xenogeneic transplant antigen.

81. The method of claim 74 wherein said cross-tolerance to an antigen results in tolerizing of CD4 helper cells to said antigen.

82. The method of claim 74 wherein said cross-tolerance to an antigen results in tolerizing of B cells to said antigen.

83. A method for enhancing clearance (immune ignorance) directed toward an apoptotic-cell-delivered antigen by a phagocyte other than a dendritic cell comprising the steps of
(a) genetically modifying said phagocyte to increase expression of an apoptotic-cell receptor capable of enhancing capture of apoptotic cells and promoting degradation of said antigen; and
(b) introducing said genetically-modified phagocyte into diseases tissue of an individual.

84. The method of claim 83 wherein said apoptotic-cell receptor is selected from the group consisting of a member of the Fc receptor family, a member of the scavenger receptor family, CD14, a member of the ABC-1 family of transporters, a member of the C-type lectin family, an integrin β subunit other than β₁, an integrin heterodimer comprising a subunit other than β₁, an integrin heterodimer comprising a chimeric β subunit other than β₁, and an integrin heterodimer comprising a mutant β subunit.

85. The method of claim 84 wherein said integrin β subunit is αᵥβ₅.

86. The method of claim 84 wherein said integrin heterodimer is αᵥβ₅.

87. The method of claim 84 wherein said integrin heterodimer comprising a chimeric β subunit comprises a wild-type α subunit and a chimeric β subunit, wherein the chimeric β subunit comprises an extracellular β₅ domain fused with a signaling domain derived from a molecule selected from the group consisting of an integrin β subunit other than β₁, a member of the Fc receptor family, a member of the scavenger receptor family, and a member of the C-type lectin family.

88. The method of claim 84 wherein said signaling domain derived from a member of the Fc receptor family is FcRγI α-chain or FcRγIIB α-chain.

89. The method of claim 84 wherein said signaling domain derived from an integrin subunit other than β₁ is that of β₂, β₃ or β₅.

90. The method of claim 83 wherein said genetically modifying said phagocyte is carried out by a method selected from the group consisting of transfection and gene transfer.

91. The method of claim 83 for the treatment of a corpse clearance diseases by the enhanced clearance of apoptotic corpses in vivo.

92. The method of claim 91 wherein said corpse clearance disease is lupus.

93. A method for enhancing cross-priming of T cells by dendritic cells or precursors thereof using an apoptotic-cell-delivered antigen comprising the steps of
(a) genetically modifying said dendritic cells or precursors thereof to increase expression of an integrin heterodimer selected from the group consisting of
i) αᵥβ₅;
ii) a heterodimer of αᵥ and a chimeric β subunit comprising an extracellular β₅ domain and a Fc FcγRI, FcγRIIA, or FcγRIII α-chain signaling domain;
iii) a heterodimer of αᵥ and a chimeric β subunit comprising an extracellular β₅ domain and an integrin β₃ or β₅ signaling domain;
iii) a β₅ subunit alone or a chimeric β subunit alone comprising an extracellular β₅ domain and an integrin β₃ or β₅ signaling domain; and
iv) a chimeric β subunit alone comprising an extracellular β₅ domain and an a Fc FcγRI, FcγRIIA, or FcγRIII α-chain signaling domain;
(b) exposing said genetically-modified phagocyte to an apoptotic cell comprising an antigen in the presence of at least one immunostimulatory exogenous factor or antigen-specific CD4 helper T cells;
wherein said dendritic cells or precursors thereof have enhanced ability to form antigen-specific CD8 cells.

94. The method of claim 93 wherein said immunostimulatory exogenous factor is at least one of CD40 ligand, TRANCE, TRAIL, OX40, or an alternate member of the TNF superfamily, thalidomide.

95. The method of claim 94 wherein said member of the TNF superfamily is TRAIL.

96. The method of claim 93 wherein said antigen is a tumor antigen and said T cells are tumor-specific T cells.

97. The method of claim 93 wherein said antigen is a viral antigen and said T cells are virus-specific or virally-infected cell specific T cells.

98. The method of claim 93 wherein said enhanced cross-priming of T cells with said antigen results in enhanced killing of tumors or virus-infected cells.

99. The method of claim 93 wherein said dendritic cells are lymphoid dendritic cells.

100. The method of claim 93 wherein said dendritic cells are myeloid dendritic cells.

101. A method for enhancing cross-tolerance to an apoptotic-cell-delivered antigen by dendritic cells or precursors thereof comprising the steps of
(a) genetically modifying said dendritic cells or precursors thereof to increase expression of an integrin heterodimer comprising
i) a heterodimer of αᵥ and a chimeric β subunit comprising an extracellular β₅ domain and a signaling β₂ domain;
ii) a chimeric β subunit alone comprising an extracellular β₅ domain and a signaling β₂ domain; or
iii) a chimeric β subunit alone comprising an extracellular β₅ domain and a signaling FcγRIIB domain;
(b) exposing said genetically-modified phagocyte to an apoptotic cell comprising an antigen in the presence of at least one immunosuppressive exogenous factor or in the absence of the combination of antigen-specific CD4 helper T cells and immunostimulatory exogenous factors;
wherein said dendritic cells have reduced ability to cross-prime T cells with said antigen.

102. The method of claim 101 wherein said immunosuppressive exogenous factor is at least one of TGF-β, IL-10, IL-4, IL-5, IL-13, FK506 or an agent that binds to FKBP12.

103. A method for treating an autoimmune disease comprising carrying out the method of claim 101.

104. The method of claim 103 wherein said autoimmune disease is psoriasis, Crohn's disease, rheumatoid arthritis, or multiple sclerosis.

105. A method for reducing the immune response to a transplant antigen comprising carrying out the method of claim 101, wherein said antigen is an allogeneic transplant antigen or a xenogeneic transplant antigen.

106. A method for stimulating the immune response in a mammalian patient to a preselected antigen to enhance the formation of antigen-specific CD8 cells comprising the steps of
a) obtaining a source of dendritic cells or precursors thereof;
b) genetically modifying said dendritic cells or precursors thereof with an apoptotic-cell receptor capable of promoting capture of apoptotic cells and enhancing cross-priming of said antigen;
c) exposing said transfected dendritic cells or precursors thereof to apoptotic cells expressing said antigen in the presence of at least one of the following compositions:
i) an agent capable of both facilitating cross-priming and maturing said dendritic cell; or
ii) the combination of at least one agent capable of facilitating cross-priming but not capable of maturing said dendritic cell, and at least one agent capable of inducing dendritic cell maturation but not capable of facilitating cross-priming;
d) optionally isolating said dendritic cells; and
e) administering said dendritic cells to a patient in need thereof.

107. The method of claim 106 wherein said dendritic cell is a myeloid dendritic cell.

108. The method of claim 106 wherein said dendritic cell is a lymphoid dendritic cell.

109. The method of claim 106 wherein said phagocyte is a human dendritic cell.

110. The method of claim 106 wherein said phagocyte is a non-human antigen presenting cell with properties similar to a dendritic cell.

111. The method of claim 106 wherein said source of dendritic cells is allogeneic cord blood, xenogeneic antigen presenting cells, bone marrow biopsy, bone marrow-derived dendritic cell precursors, isolated dendritic cell precursors, or cells obtained by leukapheresis, dendritic cells mobilized from the bone marrow to the peripheral blood.

112. The method of claim 106 wherein said agent capable of both facilitating cross-priming and maturing said phagocytic cell is a member of the TNF superfamily.

113. The method of claim 112 wherein said member of the TNF superfamily is CD40 ligand, OX40 or TRAIL.

114. The method of claim 106 wherein said agent capable of facilitating cross-priming but not capable of maturing said phagocyte is TRANCE, thalidomide or IL-12.

115. The method of claim 106 wherein said agent capable of inducing phagocyte maturation but not capable of facilitating cross-priming is monocyte conditioned medium, IL-6, TNF-α, IL-1beta or PGE₂.

116. The method of claim 106 wherein said apoptotic-cell receptor capable of promoting capture and cross-priming of T cells is selected from the group consisting of a member of the Fc receptor family, a member of the scavenger receptor family, a member of the C-type lectin family, a β integrin receptor subunit other than β₁, an integrin heterodimer other than that comprising β₁, an integrin heterodimer comprising a chimeric β subunit other than β₁, and an integrin heterodimer comprising a mutant β subunit.

117. The method of claim 116 wherein said integrin β subunit is β₅.

118. The method of claim 116 wherein said integrin heterodimer is αᵥβ₅.

119. The method of claim 116 wherein said integrin heterodimer or β subunit comprises a chimeric β subunit with an extracellular β₅ domain and an signaling domain selected from the group consisting of integrin β₃, integrin β₅, FcγRI α-chain, FcγRIIA α-chain or FcγRIII α-chain.

120. The method of claim 106 wherein said antigen is a tumor antigen and said T cells are tumor-specific T cells.

121. The method of claim 106 wherein said antigen is a viral antigen and said T cells are virus-specific or virally-infected cell specific T cells.

122. The method of claim 106 wherein said enhanced cross-priming of T cells with said antigen results in enhanced killing of tumors or virus-infected cells.

123. A method for suppressing the immune response in a mammalian patent to a preselected antigen comprising the steps of
a) obtaining a source of dendritic cells of precursors thereof;
b) genetically modifying said phagocytes with an apoptotic-cell receptor capable of promoting apoptotic cell capture, cross-presentation of an apoptotic cell-delivered antigen and promoting cross-tolerance of said antigen;
c) exposing said transfected phagocytes to apoptotic cells expressing said antigen in presence of at least one immunosuppressive exogenous factor or in the absence of the combination of CD4 helper T cells and immunostimulatory exogenous factors;
d) optionally isolating said dendritic cells; and
e) administering said dendritic cells to a patient in need thereof.

124. The method of claim 123 wherein said dendritic cell is a myeloid dendritic cell.

125. The method of claim 123 wherein said dendritic cell is a lymphoid dendritic cell.

126. The method of claim 123 wherein said source of dendritic cells or precursors thereof is allogeneic cord blood, xenogeneic antigen presenting cells, bone marrow biopsy, bone marrow-derived dendritic cell precursors, isolated dendritic cell precursors, or cells obtained by leukapheresis, dendritic cells mobilized from the bone marrow to the peripheral blood.

127. The method of claim 123 wherein said immunosuppressive exogenous factor is TGF-β IL-10, IL-4, IL-5, IL-13, FK506 or an agent that binds to FKBP12.

128. The method of claim 123 wherein said apoptotic-cell receptor capable of enhancing cross-tolerance of T cells is an integrin heterodimer with a β₂ subunit or a chimeric β subunit with an extracellular β₅ domain and an signaling domain selected from the group consisting of integrin β₂ or FcγIIB α-chain.

129. A method for treating an autoimmune disease comprising carrying out the method of claim 123.

130. The method of claim 129 wherein said autoimmune disease is psoriasis, Crohn's disease, rheumatoid arthritis, or multiple sclerosis.

131. A method for reducing the immune response to a transplant antigen comprising carrying out the method of claim 123, wherein said antigen is an allogeneic transplant antigen or a xenogeneic transplant antigen.

132. A method for increasing the expression of an αβ integrin heterodimer in a phagocyte comprising genetically modifying said phagocyte to increasing the expression of the β integrin subunit in said phagocyte.

133. The method of claim 132 wherein said β integrin subunit is native or chimeric.

134. The method of claim 133 wherein said chimeric β subunit comprises an extracellular β domain fused with a signaling domain derived from a molecule selected from the group consisting of an integrin β subunit other than β1, a member of the Fc receptor family, a member of the scavenger receptor family, and a member of the C-type lectin family.

135. The method of claim 134 wherein said signaling domain derived from a member of the Fc receptor family is the FcγRI, FcγRIIA, FcγRIIB, or FcγRIII α-chain.

136. The method of claim 134 wherein said signaling domain derived from an integrin β subunit other than β₁ is that of β₂, β₃ or β₅.

137. A method of identifying methods for altering processing of apoptotic cell-delivered antigens by a phagocytic cell comprising utilizing a 293T cell as a phagocytic cell.

138. A integrin receptor heterodimer comprising a wild-type α subunit and a chimeric β subunit, wherein the chimeric β subunit comprises an extracellular β₅ domain fused with a signaling domain derived from a molecule selected from the group consisting of an integrin β subunit other than β₁, a member of the Fc receptor family, a member of the scavenger receptor family, and a member of the C-type lectin family.

139. The integrin receptor heterodimer of claim 138 wherein said signaling domain derived from a member of the Fe receptor family is the FcγRI, FcγRIIA, FcγRIIB, or FcγRIII α-chain.

140. The integrin receptor heterodimer of claim 138 wherein said signaling domain derived from an integrin β subunit other than β₁ is that of β₂, β₃ or β₅.

141. A integrin receptor chimeric β subunit, wherein the chimeric β subunit comprises an extracellular β₅ domain fused with a signaling domain derived from a molecule selected from the group consisting of an integrin β subunit other than β₁, a member of the Fc receptor family, a member of the scavenger receptor family, and a member of the C-type lectin family.

142. The integrin receptor chimeric β subunit of claim 141 wherein said signaling domain derived from a member of the Fc receptor family is the FcγRI, FcγRIIA, FcγRIIB, or FcγRIII α-chain.

140. The integrin receptor chimeric β subunit of claim 141 wherein said signaling domain derived from an integrin β subunit other than β₁ is that of β₂, β₃ or β₅.
